(19) United States
(12) Patent Application Publication
(10) Pub. No.: US 2025/0325460 A1
(43) Pub. Date: Oct. 23, 2025

(54) HAIR TREATMENT METHOD FOR LIMITING THE CALCIUM CONTENT OF THE HAIR

(71) Applicant: L'OREAL, Paris (FR)

(72) Inventors: Adrien KAESER, Kawasaki Kanagawa (JP); Manon CHAUMONTET, SAINT-OUEN (FR)

(21) Appl. No.: 18/870,026

(22) PCT Filed: May 30, 2023
(86) PCT No.: PCT/EP2023/064380

§ 371 Date: Nov. 27, 2024

(30) Foreign Application Priority Data
Jun. 1, 2022 (FR) FR2205265

(51) Int. Cl. A61K 8/44; A61Q 5/00

(52) U.S. Cl. CPC A61K 8/44; A61Q 5/002; A61K 2800/5422; A61K 2800/5426

(57) ABSTRACT

The present invention relates to a treatment method for limiting the calcium content of the hair, comprising the application of a cosmetic composition comprising:
one or more compounds of amino acids type present in a total content of at least 0.5% by weight, with respect to the total weight of the composition, and
one or more hydroxylated (poly)carboxylic acids comprising from 2 to 8 carbon atoms, and/or their salts, present in a total content of at least 0.5% by weight, with respect to the total weight of the composition.

The present invention relates to a cosmetic treatment method for limiting the calcium content of the hair, comprising the application to said hair of a cosmetic composition comprising one or more compounds of amino acids type and one or more specific hydroxylated (poly)carboxylic acids.

Consumers over the whole world are generally in contact with highly varied sources of water which are not without impact on the hair, in particular with regard to their cosmetic properties, and/or the performance qualities of hair products.

“Mineral” waters contain, for example, variable amounts of minerals present in the form of dissolved ions, such as calcite (present in the form of calcium), dolomite (present in the form of calcium and magnesium), magnetite (present in the form of iron) and chalcanthite (present in the form of copper). “Hard” waters are also concentrated in minerals, such as calcium and magnesium, and swimming pool waters are for their part concentrated in copper salts originating from algicides used in the treatment of swimming pools.

Hair has a strong tendency to absorb these minerals and/or their metal salts because of the presence, at the surface thereof, of anionic functions which correspond in particular to the sulfonic or carboxylic functions of keratin. Furthermore, the isoelectric point of the individual hair is generally described between 3.2 and 4. The result of this is that, in everyday life, the pH of the water applied to the hair is greater than such values, which results in a negatively charged fibre.

Minerals, very often polyvalent cations, will thus be attracted and captured by this negatively charged fibre with the formation of chemical bonds, which prevent them from being released by conventional hair treatment methods. This results in a possible accumulation of minerals on the hair with the passage of time. Such an attaching depends not only on the hardness of the water, on the frequency and/or duration of exposure of the hair to the water in question but also on the nature and the length of the hair (in particular porosity and charge) and also on its state of damage.

The accumulation of these minerals and/or of their metal salts, and in particular of calcium, especially in the form of calcium ion (Ca++), can result in modifications to the hair fibre and in particular in a more or less marked modification of the cosmetic properties of the individual hair. Thus, an accumulation of calcium can result in dry hair lacking in sheen.

In addition, this accumulation can accelerate the damage caused to the hair because they catalyse oxidation/reduction reactions and generate hydroxyl radicals HO° which can be harmful to the keratin fibre, including at low contents.

In the end, this can result, in addition to a loss of sheen of the hair, in a detrimental change in the properties thereof, which can result in premature breakage.

In other words, the hair can become less resistant, more weakened, indeed even break more easily, and/or lose its sheen, due to the accumulation of calcium within the fibres (calcium ion or calcium salt).

It is all the more important to combat this phenomenon when the hair has already been sensitized, weakened and/or damaged by previous physical (repeated brushing) or chemical (dyeing, permanent wave, etc.) treatments.

There thus exists a real need to have available compositions which make it possible to combat the accumulation of calcium, especially of calcium ion, in particular resulting from minerals and metal salts dissolved in water, indeed even to make it possible to extract it from keratin fibres, in order to limit its negative impacts and to overcome all of the abovementioned disadvantages, in particular on sensitized, weakened and/or damaged hair.

This goal may be achieved by way of the employment of the composition described below.

A subject-matter of the present invention is thus a cosmetic treatment method for limiting the calcium content of the hair, comprising the application to said hair of a cosmetic composition comprising:
one or more compounds of amino acids type corresponding to the formula (I) below and/or their salts, present in a total content of at least 0.5% by weight, with respect to the total weight of the composition, and
   one or more hydroxylated (poly)carboxylic acids comprising from 2 to 8 carbon atoms, and/or their salts, present in a total content of at least 0.5% by weight, with respect to the total weight of the composition.

Another subject-matter of the invention is the use, especially cosmetic use, for limiting the calcium content of the hair, of a cosmetic composition comprising:
one or more compounds of amino acids type corresponding to the formula (I) below and/or their salts, present in a total content of at least 0.5% by weight, with respect to the total weight of the composition, and
one or more hydroxylated (poly)carboxylic acids comprising from 2 to 8 carbon atoms, and/or their salts, present in a total content of at least 0.5% by weight, with respect to the total weight of the composition.

“Limiting” the calcium content is understood to mean the act of lessening/reducing the calcium content, or even eliminating the presence of calcium in the hair, but also the act of preventing the increase in (restricting) this content.

Calcium is understood to mean especially calcium ions Ca++.

The method according to the invention is in particular a method for washing and/or conditioning the hair, especially hair which has been sensitized, weakened and/or damaged.

The method and the use according to the invention are particularly suitable for hair which has been sensitized, weakened and/or damaged, notably as a result of physical (repeated brushing) and/or chemical treatments, for example dyeing, bleaching, permanent waving and/or straightening.

The method and the use according to the invention are also particularly suitable for hair laden with calcium, especially at contents of at least 4000 ppm, better still at least 10 000 ppm.

It has been found that the use of the composition according to the invention makes it possible to significantly reduce, restrict, or even eliminate, the calcium content within the keratin fibres and consequently makes it possible to improve the resistance to breakage thereof, and therefore to strengthen them; and/or makes it possible to significantly reduce, slow down, or even halt, their loss of sheen.

Furthermore, the method according to the invention makes it possible to contribute conditioning properties to the hair, in particular a smooth feel, softness and facilitated disentangling, and this while contributing strength, body and a bulk effect to the hair.

In that which will follow, and unless otherwise indicated, the limits of a range of values are included in this range, in particular in the expressions “of between” and “ranging from . . . to . . . ”.

Furthermore, the expression “at least one” used in the present description is equivalent to the expression “one or more”, and can be substituted for it.

### Compounds of Amino Acids Type

The composition used according to the present invention comprises one or more compounds of amino acids type corresponding to the formula (I) below and/or their salts.

The compounds of amino acids type thus correspond to the formula (I):

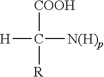

in which p is an integer equal to 1 or 2, it being understood that:
when p=1, R forms, with the nitrogen atom, a saturated heterocycle comprising from 5 to 8 ring members, preferably 5 ring members, it being possible for this ring to be substituted by one or more groups chosen from hydroxyl or (C₁-C₄)alkyl;
when p=2, R represents a hydrogen atom or a saturated, linear or branched, (C₁-C₁₂)alkyl, preferably (C₁-C₄)alkyl, group, optionally interrupted by one or more heteroatoms or groups chosen from —S—, —NH— or —C(NH)— and/or optionally substituted by one or more groups chosen from hydroxyl (—OH), amino (—NH₂), —SH, —COOH, —CONH₂ or —NH—C(NH)—NH₂.

Preferably, when p=1, R forms, with the nitrogen atom, a saturated heterocycle comprising 5 ring members, this ring not being substituted.

Preferably, p=2.

Preferably, when p=2, R represents a hydrogen atom or a saturated, linear or branched, (C₁-C₄)alkyl group, optionally interrupted by a —S— heteroatom and/or optionally substituted by one or two groups chosen from hydroxyl, amino or —NH—C(NH)—NH₂.

Preferentially, p=2 and R represents a hydrogen atom.

The compounds of amino acids type can also be a salt of a compound of formula (I).

These salts comprise the salts with organic or mineral bases, for example the salts of alkali metals, such as the lithium, sodium or potassium salts; the salts of alkaline earth metals, such as the magnesium or calcium salts, and the zinc salts.

The compounds of amino acids type can be in the form of an optical isomer of L, D or DL configuration, preferably of L configuration.

Mention may be made, as examples according to the present invention of compounds in the form of an optical isomer of L configuration, of L-proline, L-methionine, L-serine, L-arginine and L-lysine.

Preferably, the compound(s) of amino acids type according to the invention are chosen from glycine, proline, methionine, serine, arginine, lysine, their salts (in particular alkali metal, alkaline earth metal or zinc salts) and their mixtures.

Preferably, the compound(s) of amino acids type according to the invention are chosen from glycine, proline, methionine, serine, arginine, their salts and their mixtures.

Better still, the compound of amino acids type is chosen from glycine, its salts (in particular alkali metal, alkaline earth metal or zinc salts) and their mixtures.

Mention may be made, as glycine salts according to the present invention, of sodium glycinate, zinc glycinate, calcium glycinate, magnesium glycinate, manganese glycinate and potassium glycinate, preferably sodium glycinate and potassium glycinate.

Preferably, the compound of amino acid type is glycine.

The total content of compound(s) of amino acids type corresponding to the formula (I) and/or their salts, in the composition according to the invention, can range from 0.5% to 10% by weight, in particular from 0.7% to 8% by weight, better still from 0.8% to 7% by weight, with respect to the total weight of the composition.

Better still, the total content of compound(s) of amino acids type chosen from glycine, proline, methionine, serine, arginine, lysine, their salts and their mixtures in the composition according to the invention can range from 0.5% to 10% by weight, in particular from 0.7% to 8% by weight, better still from 0.8% to 7% by weight, with respect to the total weight of the composition.

Very particularly, the total content of compound(s) of amino acids type chosen from glycine, its salts and their mixtures in the composition according to the invention can range from 0.5% to 10% by weight, in particular from 0.7% to 8% by weight, better still from 0.8% to 7% by weight, with respect to the total weight of the composition. Better still, the glycine content in the composition according to the invention can range from 0.5% to 10% by weight, in particular from 0.7% to 8% by weight, better still from 0.8% to 7% by weight, with respect to the total weight of the composition.

### Hydroxy(Poly)Carboxylic Acids

The composition used according to the invention also comprises one or more hydroxylated (poly)carboxylic acids comprising from 2 to 8 carbon atoms, and/or their salts.

These (poly)acids are different from the compounds of amino acids type described above.

Said (poly)acids comprise at least one COOH group (in acid or salified form); they can thus comprise a single COOH group—the term used is then monoacid—or can comprise several of them, in particular at least 2 COOH groups (in acid or salified form), better still 2 or 3 COOH groups (in acid or salified form)—the term used is then polyacids.

(Poly)acid is understood to mean monoacids and polyacids.

They also comprise at least one OH group but can comprise several of them, in particular from 2 to 3 OH groups.

Preferably, they comprise in total from 4 to 6 carbon atoms and their hydrocarbon chain is saturated and linear.

Advantageously, the hydroxylated (poly)carboxylic acids and/or their salts comprise in total from 4 to 6 carbon atoms, from 1 to 3 OH groups and from 2 to 3 COOH groups (in acid or salified form).

The salts of these (poly)acids comprise the salts with organic or mineral bases, for example the salts of alkali metals, such as the lithium, sodium or potassium salts; the salts of alkaline earth metals, such as the magnesium or calcium salts, and the zinc salts. The alkali metal or alkaline earth metal salts are preferred, and in particular the sodium salts.

Preferably, the hydroxylated (poly)carboxylic acids or their salts are chosen from a-hydroxy acids and their salts, and in particular from lactic acid, glycolic acid, tartaric acid or citric acid, and their salts, in particular alkali metal or alkaline earth metal salts; very particularly citric acid and/or tartaric acid and also their salts, in particular alkali metal or alkaline earth metal salts, such as sodium citrate and/or sodium tartrate; better still citric acid or its salts, in particular alkali metal or alkaline earth metal salts, such as sodium citrate.

The total content of hydroxylated (poly)carboxylic acids comprising in total from 2 to 8 carbon atoms, and/or their salts, present in the composition according to the invention is at least 0.5% by weight, with respect to the total weight of the composition. This content can range from 0.5% to 10% by weight, in particular from 1% to 8% by weight, better still from 1.5% to 6% by weight, with respect to the total weight of the composition.

In particular, the total content of hydroxylated (poly)carboxylic acids comprising in total from 4 to 6 carbon atoms, from 1 to 3 OH groups and 2 or 3 COOH groups, or their salts, present in the composition according to the invention can range from 0.5% to 10% by weight, in particular from 1% to 8% by weight, better still from 1.5% to 6% by weight, with respect to the total weight of the composition.

Very particularly, the total content of hydroxylated (poly)carboxylic acids chosen from lactic acid, glycolic acid, tartaric acid or citric acid, and their salts, in particular alkali metal or alkaline earth metal salts, in the composition according to the invention can range from 0.5% to 10% by weight, in particular from 1% to 8% by weight, better still from 1.5% to 6% by weight, with respect to the total weight of the composition.

Better still, the content of citric acid and/or its salts in the composition according to the invention can range from 0.5% to 10% by weight, in particular from 1% to 8% by weight, better still from 1.5% to 6% by weight, with respect to the total weight of the composition.

### Associative Polymer

The composition used according to the invention can optionally comprise one or more associative polymers. Preferably, the associative polymers are non-ionic.

Within the meaning of the present invention, “polymer” is understood to mean any compound resulting from the polymerization by polycondensation or from the radical polymerization of monomers, one at least of which is other than an alkylene oxide, and a monofunctional compound of formula RX, R denoting an optionally hydroxylated C₁₀-C₃₀ alkyl or alkenyl group and X denoting a carboxylic acid, amine, amide, hydroxyl or ester group. All the compounds resulting solely from the simple condensation of an alkylene oxide with a fatty alcohol, a fatty ester, a fatty acid, a fatty amide or a fatty amine are in particular excluded.

Within the meaning of the present invention, “associative polymer” is understood to mean an amphiphilic polymer capable, in an aqueous medium, of reversibly combining with itself or with other molecules. It generally comprises, in its chemical structure, at least one hydrophilic region or group and at least one hydrophobic region or group.

The associative polymers according to the invention are polymers comprising at least one fatty chain comprising from 8 to 30 carbon atoms, the molecules of which are capable, in the formulation medium, of combining with one another or with molecules of other compounds. Preferably, the fatty chain comprises from 10 to 30 carbon atoms.

A particular case of associative polymers is amphiphilic polymers, that is to say polymers comprising one or more hydrophilic parts or groups which make them water-soluble and one or more hydrophobic regions or groups (comprising at least one fatty chain) by which the polymers interact and assemble together or with other molecules.

“Hydrophobic group” is understood to mean a group or a polymer having a saturated or unsaturated, linear or branched, hydrocarbon chain which can contain one or more heteroatoms, such as P, O, N or S, or a radical having a perfluoro or silicone chain. When it denotes a hydrocarbon group, the hydrophobic group comprises at least 10 carbon atoms, preferably from 10 to 30 carbon atoms, in particular from 12 to 30 carbon atoms and preferentially from 18 to 30 carbon atoms. Preferentially, the hydrophobic hydrocarbon group originates from a monofunctional compound.

By way of example, the hydrophobic group can result from a fatty alcohol, such as stearyl alcohol, dodecyl alcohol or decyl alcohol, or else from a polyalkylenated fatty alcohol, such as Steareth-100. It can also denote a hydrocarbon polymer, such as, for example, polybutadiene.

Within the meaning of the present invention, “fatty chain” is understood to mean a linear or branched alkyl or alkenyl chain comprising at least 8 carbon atoms, preferably from 8 to 30 carbon atoms and better still from 10 to 22 carbon atoms.

Within the meaning of the present invention, “fatty compound”, such as, for example, a fatty alcohol, a fatty acid or a fatty amide, is understood to mean a compound comprising, in its main chain, at least one saturated or unsaturated hydrocarbon chain, such as an alkyl or alkenyl chain, comprising at least 8 carbon atoms, preferably from 8 to 30 carbon atoms and better still from 10 to 22 carbon atoms.

Mention may in particular be made, among the the anionic associative polymers, of:
(A) those comprising at least one hydrophilic unit and at least one allyl ether unit having a fatty chain; and more particularly those:
   the hydrophilic unit of which is constituted by an ethylenic unsaturated anionic monomer, more particularly still by a vinylcarboxylic acid and very particularly by an acrylic acid or a methacrylic acid or the mixtures of these; and the allyl ether unit having a fatty chain of which corresponds to the monomer of following formula (I′): CH₂═C(R′)—CH₂OBₙR
   in which R′ denotes H or CH₃, B denotes an ethyleneoxy radical, n ranges from 0 to 100 and R denotes a hydrocarbon radical chosen from the alkyl, arylalkyl, aryl, alkylaryl and cycloalkyl radicals, comprising from 8 to 30 carbon atoms, preferably from 10 to 24 carbon atoms and more particularly still from 12 to 18 carbon atoms.

Preferably, R′ denotes H, n=10 and R denotes a stearyl (C₁₈) radical.

Preference is particularly given, among these anionic associative polymers, to the polymers formed from 20% to 60% by weight of acrylic acid and/or of methacrylic acid, from 5% to 60% by weight of C₁-C₄ alkyl (meth)acrylates, from 2% to 50% by weight of allyl ether having a fatty chain of formula (I′) and from 0% to 1% by weight of a crosslinking agent which is preferably a copolymerizable polyethylenic unsaturated monomer, such as diallyl phthalate, allyl (meth)acrylate, divinylbenzene, (poly)ethylene glycol dimethacrylate or methylenebisacrylamide.

Preference is very particularly given to crosslinked terpolymers of methacrylic acid, of ethyl acrylate and of polyethylene glycol (10 EO) stearyl alcohol ether (Steareth-10), in particular that sold by BASF under the name Salcare SC80, which is a 30% aqueous emulsion of a crosslinked terpolymer of methacrylic acid, of ethyl acrylate and of Steareth-10 allyl ether (40/50/10) with the INCI name Steareth-10 Allyl Ether/Acrylates Copolymer;
(B) those comprising at least one hydrophilic unit of unsaturated olefinic carboxylic acid type and at least one hydrophobic unit of (C₁₀-C₃₀)alkyl ester of unsaturated carboxylic acid type.

These polymers are preferably chosen from those for which the hydrophilic unit of unsaturated olefinic carboxylic acid type corresponds to the monomer of following formula (II):

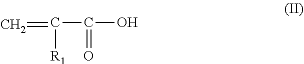

in which R₁ denotes H, CH₃ or C₂H₅, and for which the hydrophobic unit of (C₁₀-C₃₀)alkyl ester of unsaturated carboxylic acid type corresponds to the monomer of following formula (III):

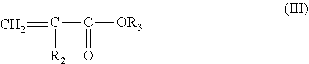

in which R₂ denotes H, CH₃ or C₂H₅ and R₃ denotes a C₁₀-C₃₀, preferably C₁₂-C₂₂, alkyl radical.

(C₁₀-C₃₀)alkyl esters of unsaturated carboxylic acids in accordance with the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Use will more particularly be made, among these anionic associative polymers, of polymers formed from a mixture of monomers comprising: (i) (meth)acrylic acid, (ii) an ester of formula (III) described above and in which R₂ denotes H or CH₃ and R₃ denotes an alkyl radical having from 12 to 22 carbon atoms, and optionally (iii) a crosslinking agent which is a well-known copolymerizable polyethylenic unsaturated monomer, such as diallyl phthalate, allyl (meth)acrylate, divinylbenzene, (poly)ethylene glycol dimethacrylate and methylenebisacrylamide.

Preference is more particularly given, among anionic associative polymers of this type, to those constituted of from 95% to 60% by weight of (meth)acrylic acid, 4% to 40% by weight of C₁₀-C₃₀ alkyl acrylate and 0% to 6% by weight of crosslinking polymerizable monomer, or else to those constituted of from 98% to 96% by weight of (meth)acrylic acid, 1% to 4% by weight of C₁₀-C₃₀ alkyl acrylate and 0.1% to 0.6% by weight of crosslinking polymerizable monomer, such as those described above.

Mention may in particular be made of the products sold by Lubrizol under the trade names Pemulen TR1, Pemulen TR2, Carbopol 1382, Carbopol ETD 2020, Carbopol Ultrez 20, Carbopol Ultrez 21, with the INCI name Acrylates/C₁₀₋₃₀ Alkyl Acrylate Crosspolymer, and more preferentially still of Pemulen TR1 and Carbopol 1382;
(C) maleic anhydride/C₃₀-C₃₈ α-olefin/alkyl maleate terpolymers, such as the maleic anhydride/C₃₀-C₃₈ α-olefin/isopropyl maleate copolymer, in particular that sold under the name Performa V 1608 by Newphase Technologies (INCI name: C₃₀₋₃₈ Olefin/Isopropyl Maleate/MA Copolymer);
(D) acrylic terpolymers comprising (a) from 20% to 70% by weight of an α,β-monoethylenically unsaturated carboxylic acid, (b) from 20% to 80% by weight of a non-surface-active α,β-monoethylenically unsaturated monomer other than (a), and (c) from 0.5% to 60% by weight of a non-ionic monourethane which is the reaction product of a monohydric surfactant with a monoethylenically unsaturated monoisocyanate.

Mention may in particular be made of the methacrylic acid/methyl acrylate/dimethyl(meta-isopropenyl)benzyl isocyanate of ethoxylated (40 EO) behenyl alcohol terpolymer, in particular as a 25% aqueous dispersion, such as the product Viscophobe DB1000 sold by Amerchol (Dow Chemical) having the INCI name Polyacrylate-3;
(E) copolymers comprising among their monomers (i) an α,β-monoethylenically unsaturated carboxylic acid, such as acrylic or methacrylic acid, and (ii) an ester of α,β-monoethylenically unsaturated carboxylic acid, in particular acrylic or methacrylic acid, and of fatty alcohol, in particular C₈-C₃₂ fatty alcohol, which is oxyalkylenated, in particular comprising from 2 to 100 mol of ethylene oxide, in particular from 4 to 50, indeed even from 10 to 40 EO.

Mention may in particular be made, as monomers, of behenyl or stearyl (meth)acrylate comprising from 10 to 40 EO, in particular from 18 to 30 EO. Preferentially, these compounds also comprise, as monomer, an ester of α,β-monoethylenically unsaturated carboxylic acid and of C₁-C₄ alcohol, in particular a C₁-C₄ alkyl (meth)acrylate.

Preferably, these copolymers comprise at least one (meth)acrylic acid monomer, at least one C₁-C₄ alkyl (meth)acrylate monomer and at least one C₈-C₃₂ alkyl (meth)acrylate monomer which is oxyethylenated, comprising from 2 to 100 mol EO, in particular from 4 to 50 EO, indeed even from 10 to 40 EO.

Mention may be made, by way of examples, of Aculyn 22 sold by Rohm and Haas, which is an oxyalkylenated methacrylic acid/ethyl acrylate/stearyl methacrylate terpolymer (INCI name: Acrylates/Steareth-20 Methacrylate Copolymer), or also of Aculyn 28 sold by Rohm and Haas, which is an oxyalkylenated methacrylic acid/ethyl acrylate/behenyl methacrylate terpolymer (INCI name: Acrylates/Beheneth-25 Methacrylate Copolymer), as well as of the Novethix L-10 Polymer sold by Lubrizol;
(F) associative polymers comprising at least one ethylenically unsaturated monomer having a sulfonic group, in the free or partially or completely neutralized form, and comprising at least one hydrophobic part.

Mention may more especially be made, among the polymers of this type, of:
crosslinked or non-crosslinked, neutralized or non-neutralized, copolymers comprising from 15% to 60% by weight of AMPS (2-acrylamido-2-methylpropanesulfonic acid or salt) units and from 40% to 85% by weight of (C₈-C₁₆)alkyl (meth)acrylate units with respect to the polymer, such as are described in Patent Application EP-A-750 899;
terpolymers comprising from 10 mol % to 90 mol % of acrylamide units, from 0.1 mol % to 10 mol % of AMPS units and from 5 mol % to 80 mol % of n-(C₆-C₈)alkylacrylamide units, such as those described in Patent U.S. Pat. No. 5,089,578;
copolymers of completely neutralized AMPS and of dodecyl methacrylate, and also copolymers of AMPS and of n-dodecylmethacrylamide, which are non-crosslinked and crosslinked;
copolymers constituted of AMPS units and of steareth-25 methacrylate units, such as Aristoflex HMS® sold by Clariant (INCI name: Ammonium Acryloyldimethyltaurate/Steareth-25 Methacrylate Crosspolymer), or beheneth-25 methacrylate units, such as Aristoflex HMB (INCI name: Ammonium Acryloyldimethyltaurate/Beheneth-25 Methacrylate Crosspolymer) sold by Clariant, or also steareth-8 methacrylate units, such as Aristoflex SNCO from Clariant (INCI name: Ammonium Acryloyldimethyltaurate/Steareth-8 Methacrylate Copolymer);
(G) associative polymers comprising at least one vinyllactam monomer and at least one α,β-monoethylenically unsaturated carboxylic acid monomer, such as terpolymers of vinylpyrrolidone, of acrylic acid and of C₁-C₂₀ alkyl methacrylate, for example lauryl methacrylate, such as that sold by ISP under the name Acrylidone® LM (INCI name: VP/Acrylates/Lauryl Methacrylate Copolymer).

Mention may be made, among the cationic associative polymers, of:
(A′) cationic associative polyurethanes, which can be represented by the following general formula (Ia): R—X—(P)ₙ—[L-(Y)ₘ]ᵣ-L′—(P′)ₚ—X′—R′
in which:
R and R′, which are identical or different, represent a hydrophobic group or a hydrogen atom;
X and X′, which are identical or different, represent a group comprising an amine function carrying or not carrying a hydrophobic group, or also the group L″;
L, L′ and L″, which are identical or different, represent a group derived from a diisocyanate;
P and P′, which are identical or different, represent a group comprising an amine function carrying or not carrying a hydrophobic group;
Y represents a hydrophilic group;
r is an integer of between 1 and 100 inclusive, preferably between 1 and 50 inclusive and in particular between 1 and 25 inclusive;
n, m and p are each, independently of one another, between 0 and 1000 inclusive; the molecule containing at least one protonated or quaternized amine function and at least one hydrophobic group.

Preferably, the only hydrophobic groups are the groups R and R′ at the chain ends.

A preferred family of cationic associative polyurethanes is that corresponding to the formula (Ia) described above in which:
R and R′ both independently represent a hydrophobic group,
X and X′ each represent a group L″,
n and p are integers which have values between 1 and 1000 inclusive, and
L, L′, L″, P, P′, Y and m have the meanings given above.

Another preferred family of cationic associative polyurethanes is that corresponding to the formula (Ia) above in which:
n=p=0 (the polymers do not comprise units derived from a monomer having an amine function, which is incorporated in the polymer during the polycondensation), the protonated amine functions result from the hydrolysis of excess isocyanate functions, at the chain end, followed by the alkylation of the primary amine functions formed with alkylating agents having a hydrophobic group, that is to say compounds of type RQ or R′Q, in which R and R′ are as defined above and Q denotes a leaving group, such as a halide, a sulfate, and the like.

Yet another preferred family of cationic associative polyurethanes is that corresponding to the formula (Ia) above in which:
R and R′ both independently represent a hydrophobic group,
X and X′ both independently represent a group comprising a quaternary amine,
n=p=0, and
L, L′, Y and m have the meanings indicated above.

The number-average molecular weight (Mn) of the cationic associative polyurethanes is of preferably between 400 and 500 000 inclusive, in particular between 1000 and 400 000 inclusive and ideally between 1000 and 300 000 inclusive.

Preferentially, the hydrocarbon group originates from a monofunctional compound. By way of example, the hydrophobic group can result from a fatty alcohol, such as stearyl alcohol, dodecyl alcohol or decyl alcohol. It can also denote a hydrocarbon polymer, such as, for example, polybutadiene.

When X and/or X′ denote(s) a group comprising a tertiary or quaternary amine, X and/or X′ can represent one of the following formulae:

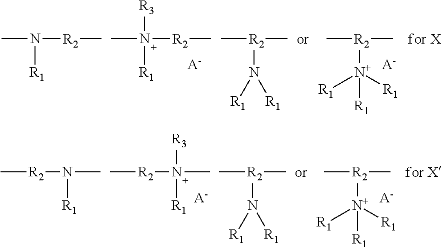

in which:
R₂ represents a linear or branched alkylene radical having from 1 to 20 carbon atoms, comprising or not comprising a saturated or unsaturated ring, or an arylene radical, it being possible for one or more of the carbon atoms to be replaced with a heteroatom chosen from N, S, O or P;
R₁ and R₃, which are identical or different, denote a linear or branched C₁-C₃₀ alkyl or alkenyl radical or an aryl radical, it being possible for one at least of the carbon atoms to be replaced with a heteroatom chosen from N, S, O or P; A⁻ is a physiologically acceptable anionic counterion, such as a halide, for instance a chloride or bromide, or a mesylate.

The groups L, L′ and L″ represent a group of formula:

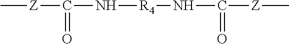

in which:
Z represents —O—, —S— or —NH—; and
R₄ represents a linear or branched alkylene radical having from 1 to 20 carbon atoms, comprising or not comprising a saturated or unsaturated ring, an arylene radical, it being possible for one or more of the carbon atoms to be replaced with a heteroatom chosen from N, S, O and P.

The groups P and P′ comprising an amine function can represent at least one of the following formulae:

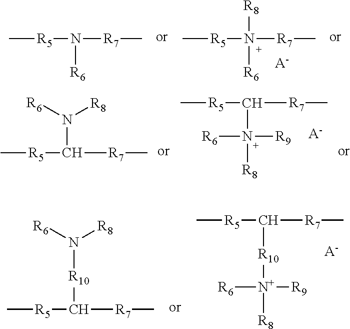

in which:
R₅ and R₇ have the same meanings as R₂ defined above;
R₆, R₈ and R₉ have the same meanings as R₁ and R₃ defined above; R₁₀ represents a linear or branched alkylene group which is optionally unsaturated and which can contain one or more heteroatoms chosen from N, O, S and P; and
A⁻ is a physiologically acceptable anionic counterion, such as a halide, for instance chloride or bromide, or mesylate.

As regards the meaning of Y, hydrophilic group is understood to mean a polymeric or non-polymeric water-soluble group.

Mention may be made, by way of example, when a polymer is not concerned, of ethylene glycol, diethylene glycol and propylene glycol.

Mention may be made, when a hydrophilic polymer is concerned, by way of example, of polyethers, sulfonated polyesters, sulfonated polyamides or a mixture of these polymers. Preferentially, the hydrophilic compound is a polyether and in particular a poly(ethylene oxide) or poly(propylene oxide).

The cationic associative polyurethanes of formula (Ia) according to the invention are formed from diisocyanates and from various compounds having functions comprising labile hydrogen. The functions comprising labile hydrogen can be alcohol, primary or secondary amine or thiol functions giving, after reaction with the diisocyanate functions, respectively polyurethanes, polyureas and polythioureas.

The term “polyurethanes” of the present invention encompasses these three types of polymers, namely polyurethanes proper, polyureas and polythioureas, and also copolymers of these.

A first type of compounds participating in the preparation of the polyurethane of formula (Ia) is a compound comprising at least one unit having an amine function.

This compound can be multifunctional but preferentially the compound is difunctional, that is to say that, according to a preferential embodiment, this compound comprises two labile hydrogen atoms carried, for example, by a hydroxyl, primary amine, secondary amine or thiol function. A mixture of multifunctional and difunctional compounds in which the percentage of multifunctional compounds is low can also be used.

As indicated above, this compound can comprise more than one unit having an amine function. It then concerns a polymer carrying a repetition of the unit having an amine function.

Compounds of this type can be represented by one of the following formulae: HZ—(P)ₙ—ZH or HZ—(P′)ₚ—ZH, in which Z, P, P′, n and p are as defined above.

Mention may be made, by way of example, of N-methyldiethanolamine, N-(tert-butyl)diethanolamine and N-sulfoethyldiethanolamine.

The second compound participating in the preparation of the polyurethane of formula (Ia) is a diisocyanate corresponding to the formula: O═C═N—R₄—N═C═O, in which R₄ is as defined above.

Mention may be made, by way of example, of methylenediphenyl diisocyanate, methylenecyclohexane diisocyanate, isophorone diisocyanate, toluene diisocyanate, naphthalene diisocyanate, butane diisocyanate or hexane diisocyanate.

A third compound participating in the preparation of the polyurethane of formula (Ia) is a hydrophobic compound intended to form the terminal hydrophobic groups of the polymer of formula (Ia).

This compound is constituted of a hydrophobic group and of a function having labile hydrogen, for example a hydroxyl, primary or secondary amine or thiol function.

By way of example, this compound can be a fatty alcohol, such as stearyl alcohol, dodecyl alcohol or decyl alcohol. When this compound comprises a polymeric chain, it can, for example, concern a-hydroxylated hydrogenated polybutadiene.

The hydrophobic group of the polyurethane of formula (Ia) can also result from the quaternization reaction of the tertiary amine of the compound comprising at least one tertiary amine unit. Thus, the hydrophobic group is introduced by the quaternizing agent. This quaternizing agent is a compound of RQ or R′Q type, in which R and R′ are as defined above and Q denotes a leaving group, such as a halide, a sulfate, and the like.

The cationic associative polyurethane can additionally comprise a hydrophilic block.

This block is provided by a fourth type of compound participating in the preparation of the polymer. This compound can be multifunctional. It is preferably difunctional. It is also possible to have a mixture in which the percentage of multifunctional compound is low.

The functions having labile hydrogen are alcohol, primary or secondary amine or thiol functions. This compound can be a polymer terminated at the ends of the chains with one of these functions having labile hydrogen.

Mention may be made, by way of example, when polymers are not concerned, of ethylene glycol, diethylene glycol and propylene glycol.

Mention may be made, when a hydrophilic polymer is concerned, by way of example, of polyethers, sulfonated polyesters, sulfonated polyamides or a mixture of these polymers. Preferentially, the hydrophilic compound is a polyether and in particular a poly(ethylene oxide) or poly(propylene oxide).

The hydrophilic group termed Y in the formula (Ia) is optional. Specifically, the units having quaternary or protonated amine function can suffice to contribute the solubility or the water-dispersibility required for this type of polymer in an aqueous solution.

Although the presence of a hydrophilic group Y is optional, it is preferable, however, for cationic associative polyurethanes to comprise such a group.
(B′) quaternized cellulose derivatives, and in particular:
i) quaternized celluloses modified with groups comprising at least one fatty chain, such as linear or branched alkyl, linear or branched arylalkyl or linear or branched alkylaryl groups comprising at least 8 carbon atoms, or mixtures of these;
ii) quaternized hydroxyethylcelluloses modified with groups comprising at least one fatty chain, such as linear or branched alkyl, linear or branched arylalkyl or linear or branched alkylaryl groups comprising at least 8 carbon atoms, or mixtures of these;
iii) hydroxyethylcelluloses of formula (Ib):

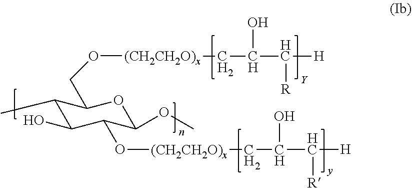

in which:
R and R′, which are identical or different, represent an ammonium group —RₐR_{b}R_{c}N⁺Q⁻ in which Rₐ, R_{b} and R_{c}, which are identical or different, represent a hydrogen atom or a linear or branched C₁-C₃₀, preferentially C₁-C₂₀, alkyl group, such as methyl or dodecyl; and Q⁻ represents an anionic counterion, such as a halide, for instance a chloride or bromide; and n, x and y, which are identical or different, represent an integer of between 1 and 10 000.

The alkyl radicals carried by the above quaternized celluloses i) or hydroxyethylcelluloses ii) preferably comprise from 8 to 30 carbon atoms. The aryl radicals preferably denote phenyl, benzyl, naphthyl or anthryl groups.

It is also possible to indicate, as examples of quaternized alkylhydroxyethylcelluloses having C₈-C₃₀ fatty chains, the product Quatrisoft LM 200® sold by Amerchol/Dow Chemical (INCI name: Polyquaternium-24) and the products Crodacel QM® (INCI name: PG-Hydroxyethylcellulose Cocodimonium Chloride), Crodacel QL® (C₁₂ alkyl) (INCI name: PG-Hydroxyethylcellulose Lauryldimonium Chloride) and Crodacel QS® (C₁₈ alkyl) (INCI name: PG-Hydroxyethylcellulose Stearyldimonium Chloride), which are sold by Croda. Mention may also be made of the hydroxyethylcelluloses of formula (Ib) in which R represents a trimethylammonium halide and R′ represents a dimethyldodecylammonium halide; more preferentially, R represents trimethylammonium chloride —(CH₃)₃N⁺Cl⁻ and R′ represents dimethyldodecylammonium chloride —(CH₃)₂(C₁₂H₂₅)N⁺Cl⁻. Polymers of this type are known under the trade name Softcat Polymer SL®, such as SL-100, SL-60, SL-30 and SL-5, from Amerchol/Dow Chemical, with the INCI name Polyquaternium-67. More particularly, the polymers of formula (Ib) are those, the viscosity of which is between 2000 and 3000 cPs inclusive. Preferentially, the viscosity is of between 2700 and 2800 cPs inclusive. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs.
(C′) cationic polyvinyllactams, in particular those comprising:
a) at least one monomer of vinyllactam or alkylvinyllactam type;
b) at least one monomer of following structure (Ic) or (IIc):

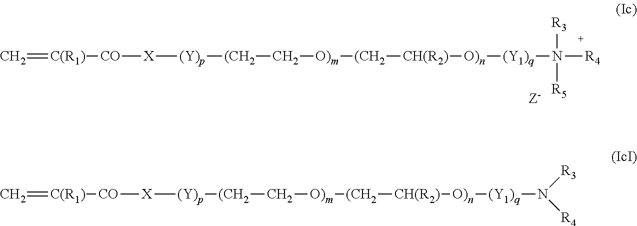

in which:
X denotes an oxygen atom or an NR₆ radical,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of one another, a hydrogen atom, a linear or branched C₁-C₃₀ alkyl radical or a radical of formula (IIIc):

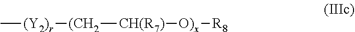

in which:
   Y, Y₁ and Y₂ denote, independently of one another, a linear or branched C₂-C₁₆ alkylene radical,
R₇ denotes a hydrogen atom, a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of one another, either the value zero or the value 1,
m and n denote, independently of each other, an integer ranging from 0 to 100 inclusive,
x denotes an integer ranging from 1 to 100 inclusive,
Z denotes an anionic counterion of an organic or mineral acid, such as halide, for instance chloride or bromide, or mesylate;
with the proviso that:
at least one of the substituents R₃, R₄, R₅ or R₈ denotes a linear or branched C₉-C₃₀ alkyl radical,
   if m or n is other than zero, then q is equal to 1,
   if m or n is equal to zero, then p or q is equal to 0.

The cationic poly(vinyllactam) polymers according to the invention can be crosslinked or noncrosslinked and can also be block polymers.

Preferably, the counterion Z⁻ of the monomers of formula (Ic) is chosen from halide ions, phosphate ions, the methosulfate ion and the tosylate ion.

Preferably, R₃, R₄ and R₅ denote, independently of one another, a hydrogen atom or a linear or branched C₁-C₃₀ alkyl radical.

More preferentially, the monomer b) is a monomer of formula (Ic) for which, preferentially, m and n are equal to 0.

The vinyllactam or alkylvinyllactam monomer is preferably a compound of structure (IVc):

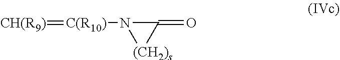

in which s denotes an integer ranging from 3 to 6; R₉ denotes a hydrogen atom or a linear or branched C₁-C₅ alkyl radical and R₁₀ denotes a hydrogen atom or a linear or branched C₁-C₅ alkyl radical, with the proviso that one at least of the radicals R₉ and R₁₀ denotes a hydrogen atom.

More preferentially still, the monomer (IVc) is vinylpyrrolidone.

The cationic poly(vinyllactam) polymers according to the invention can also contain one or more additional monomers, preferably cationic or non-ionic monomers.

Mention may be made, as compounds which are particularly preferred, of the following terpolymers comprising at least:
a) one monomer of formula (IVc),
b) one monomer of formula (Ic) in which p=1, q=0, R₃ and R₄ denote, independently of each other, a hydrogen atom or a C₁-C₅ alkyl radical and R₅ denotes a linear or branched C₉-C₂₄ alkyl radical, and
c) one monomer of formula (IIc) in which R₃ and R₄ denote, independently of each other, a hydrogen atom or a linear or branched C₁-C₅ alkyl radical.

More preferentially still, use will be made of terpolymers comprising, by weight, from 40% to 95% of monomer (a), from 0.1% to 55% of monomer (c) and from 0.25% to 50% of monomer (b). Such polymers are in particular described in Patent Application WO-00/68282.

Use may in particular be made, as cationic poly(vinyllactam) polymers according to the invention, of:
vinylpyrrolidone/dimethylaminopropylmethacrylamide/dodecyldimethylmethacryla midopropylammonium tosylate terpolymers,
vinylpyrrolidone/dimethylaminopropylmethacrylamide/cocoyldimethylmethacrylami dopropylammonium tosylate terpolymers,
vinylpyrrolidone/dimethylaminopropylmethacrylamide/lauryldimethylmethacrylamid opropylammonium tosylate or chloride terpolymers.

The vinylpyrrolidone/dimethylaminopropylmethacrylamide/lauryldimethylmethacrylamid opropylammonium chloride terpolymer is in particular sold by ISP under the name Styleze W10® or Styleze W20L® (INCI name: Polyquaternium-55).

The weight-average molecular weight (Mw) of the cationic poly(vinyllactam) polymers is preferably of between 500 and 20 000 000, more particularly between 200 000 and 2 000 000 and preferentially between 400 000 and 800 000.
(D′) the cationic polymers obtained by polymerization of a mixture of monomers comprising one or more vinyl monomers substituted with one or more amino groups, one or more hydrophobic non-ionic vinyl monomers and one or more associative vinyl monomers, such as are described in Patent Application WO 2004/024779.

Mention may more particularly be made, among these polymers, of the products of the polymerization of a mixture of monomers comprising:
a di(C₁-C₄ alkyl)amino(C₁-C₅ alkyl) methacrylate,
one or more C₁-C₃₀ alkyl esters of (meth)acrylic acid,
a polyethoxylated (20-25 mol of ethylene oxide unit) C₁₀-C₃₀ alkyl methacrylate,
a 30/5 polyethylene glycol/polypropylene glycol allyl ether,
a hydroxy(C₂-C₆ alkyl) methacrylate, and
an ethylene glycol dimethacrylate.

Such a polymer is, for example, the compound sold by Lubrizol under the name Carbopol Aqua CCO and which corresponds to the INCI name Polyacrylate-1 Crosspolymer.

The non-ionic associative polymers are preferably chosen from, alone or as a mixture:
(1) Celluloses modified with groups comprising at least one fatty chain, in particular a C₈-C₃₂ and better still C₁₄-C₂₈ alkyl chain; preferably from:
hydroxyethylcelluloses modified with groups comprising at least one fatty chain, in particular a C₈-C₃₂ and better still C₁₄-C₂₈ alkyl chain, such as alkyl, arylalkyl or alkylaryl groups, or their mixtures, and in which the alkyl groups are preferably C₈-C₂₂ alkyl groups, such as the cetylhydroxyethylcellulose sold in particular under the reference Natrosol Plus Grade 330 CS (C₁₆ alkyls) by Ashland or the product Polysurf 67CS sold by Ashland,
hydroxyethylcelluloses modified with polyalkylene glycol alkylphenol ether groups, such as the product Amercell Polymer HM-1500 (polyethylene glycol (15) nonylphenol ether) sold by Amerchol,
and their mixtures.
(2) Hydroxypropyl guars modified with groups comprising at least one fatty chain, in particular a C₈-C₃₂ and better still C₁₄-C₂₈ alkyl chain, such as the product Esaflor HM 22 (C₂₂ alkyl chain) sold by Lamberti and the products RE210-18 (C₁₄ alkyl chain) and RE205-1 (C₂₀ alkyl chain) sold by Solvay.
(3) Copolymers of vinylpyrrolidone and of hydrophobic monomers having a fatty chain, in particular a C₈-C₃₂ and better still C₁₄-C₂₈ alkyl chain. Mention may be made, by way of example, of:
   the vinylpyrrolidone/hexadecene copolymer and in particular the product Antaron V216 or Ganex V216 sold by ISP;
   the vinylpyrrolidone/eicosene copolymer and in particular the product Antaron V220 or Ganex V220 sold by ISP.
(4) Copolymers of C₁-C₆ alkyl methacrylates or acrylates and of amphiphilic monomers comprising at least one fatty chain, in particular a C₈-C₃₂ and better still C₁₄-C₂₈ alkyl chain, such as, for example, the oxyethylenated methyl acrylate/stearyl acrylate copolymer sold by Goldschmidt under the name Antil 208.
(5) Copolymers of hydrophilic methacrylates or acrylates and of hydrophobic monomers comprising at least one fatty chain, in particular a C₈-C₃₂ and better still C₁₄-C₂₈ alkyl chain, such as, for example, the polyethylene glycol methacrylate/lauryl methacrylate copolymer.
(6) polyether polyurethanes comprising, in their chain, both hydrophilic blocks usually of polyoxyethylenated nature and hydrophobic blocks which can be aliphatic sequences alone and/or cycloaliphatic and/or aromatic sequences.
(7) Polymers having an aminoplast ether backbone possessing at least one fatty chain, in particular a C₈-C₃₂ and better still C₁₄-C₂₈ alkyl chain, such as the Pure Thix compounds provided by Suid-Chemie.

Preferably, the polyether polyurethanes comprise at least two lipophilic hydrocarbon chains having from 8 to 30 carbon atoms, separated by a hydrophilic block, it being possible for the hydrocarbon chains to be pendent chains or chains at the end of the hydrophilic block. In particular, it is possible for one or more pendent chains to be envisaged. In addition, the polymer can comprise a hydrocarbon chain at one end or at both ends of a hydrophilic block.

The polyether polyurethanes can be multiblock, in particular in triblock form. The hydrophobic blocks can be at each end of the chain (for example: triblock copolymer having a hydrophilic central block) or distributed both at the ends and in the chain (for example, multiblock copolymer). These same polymers can also be graft polymers or star polymers.

The non-ionic polyether polyurethanes having a fatty chain can be triblock copolymers, the hydrophilic block of which is a polyoxyethylene chain comprising from 50 to 1000 oxyethylene groups. The non-ionic polyether polyurethanes comprise a urethane bond between the hydrophilic blocks, hence the origin of the name.

By extension, the non-ionic polyether polyurethanes having a fatty chain also include those, the hydrophilic blocks of which are bonded to the lipophilic blocks via other chemical bonds.

Mention may be made, as examples of non-ionic polyether polyurethanes having a fatty chain which can be used in the invention, of Rheolate 205® having a urea function, sold by Rheox, or also Rheolate® 208, 204 or 212, and also Acrysol RM 184®. Mention may also be made of the product Elfacos T210® having a C₁₂-C₁₄ alkyl chain and the product Elfacos T212® having a C₁₈ alkyl chain from Akzo. The product DW 1206B© from Rohm and Haas having a C₂₀ alkyl chain and having a urethane bond, provided at a solids content of 20% in water, can also be used.

Use may also be made of solutions or dispersions of these polymers, in particular in water or in an aqueous/alcoholic medium. Mention may be made, as examples of such polymers, of Rheolate® 255, Rheolate® 278 and Rheolate® 244, sold by Rheox. Use may also be made of the products DW 1206F and DW 1206J, provided by Rohm and Haas.

The polyether polyurethanes which can be used according to the invention are in particular those described in the paper by G. Fonnum, J. Bakke and Fk. Hansen—Colloid Polym. Sci., 271, 380-389 (1993).

More particularly still, it is preferred to use a polyether polyurethane capable of being obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyether polyurethanes are sold in particular by Rohm and Haas under the names Aculyn 46® and Aculyn 44®[Aculyn 46® is a polycondensate of polyethylene glycol having 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol having 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the composition comprises one more or non-ionic associative polymers, preferentially chosen from polyether polyurethanes.

Preferably, the associative polymer(s) are present in the composition in a total content ranging from 0.01% to 10% by weight, preferentially from 0.05% to 5% by weight, more preferentially from 0.1% to 1.5% by weight, with respect to the total weight of the composition.

Preferably, the non-ionic associative polymer(s) are present in the composition in a total content ranging from 0.01% to 10% by weight, preferentially from 0.05% to 5% by weight, more preferentially from 0.1% to 1.5% by weight, with respect to the total weight of the composition.

Preferably, the non-ionic associative polymer(s) chosen from polyether polyurethanes are present in the composition in a total content ranging from 0.01% to 10% by weight, preferentially from 0.05% to 5% by weight, more preferentially from 0.1% to 1.5% by weight, with respect to the total weight of the composition.

### Silicones

The composition used according to the invention can optionally comprise one or more silicones, which can in particular be chosen from aminated silicones, non-aminated silicones and their mixtures.

The composition according to the invention can thus comprise one or more non-aminated silicones, which can be solid or liquid, preferably liquid (at 25° C., 1 atm), and volatile or non-volatile.

The non-aminated silicones which can be used can be soluble or insoluble in the composition according to the invention; they can be provided in the oil, wax, resin or gum form; silicone oils and gums are preferred.

Silicones are in particular described in detail in the work by Walter Noll, Chemistry and Technology of Silicones (1968), Academic Press.

The volatile silicones can be chosen from those possessing a boiling point of between 60° C. and 260° C. (at atmospheric pressure) and in particular from: i) cyclic polydialkylsiloxanes comprising from 3 to 7 and preferably from 4 to 5 silicon atoms, such as
octamethylcyclotetrasiloxane (D4) and decamethylcyclopentasiloxane (D5).

Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia;
cyclocopolymers of the dimethylsiloxane/methylalkylsiloxane type with the chemical structure:

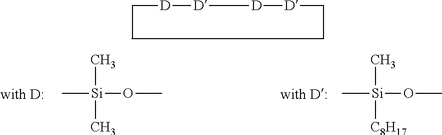

Mention may be made of Volatile Silicone FZ 3109 sold by Union Carbide;
mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;
ii) linear polydialkylsiloxanes having from 2 to 9 silicon atoms, which generally possess a viscosity of less than or equal to 5.10⁻⁶ m²/s at 25° C., such as decamethyltetrasiloxane.

Other silicones coming within this category are described in the paper published in Cosmetics and Toiletries, Vol. 91, January 76, pages 27-32—Todd & Byers, Volatile Silicone Fluids for Cosmetics; mention may be made of the product sold under the name SH 200 by Toray Silicone.

Mention may be made, among the non-volatile silicones, alone or as a mixture, of polydialkylsiloxanes and in particular polydimethylsiloxanes (PDMS or dimethicone), polydiarylsiloxanes, polyalkylarylsiloxanes, silicone gums and resins, and also non-aminated organopolysiloxanes (or organomodified polysiloxanes, or also organomodified silicones), which are polysiloxanes comprising, in their structure, one or more non-aminated organofunctional groups, generally attached via a hydrocarbon group, and preferably chosen from aryl groups, alkoxy groups and polyoxyethylene and/or polyoxypropylene groups.

The organomodified silicones can be polydiarylsiloxanes, in particular polydiphenylsiloxanes, and polyalkylarylsiloxanes functionalized with the abovementioned organofunctional groups. The polyalkylarylsiloxanes are particularly chosen from linear and/or branched polydimethyl/methylphenylsiloxanes or polydimethyl/diphenylsiloxanes.

Mention may be made, among the organomodified silicones, of the organopolysiloxanes comprising:
polyoxyethylene and/or polyoxypropylene groups optionally comprising C₆-C₂₄ alkyl groups, such as dimethicone copolyols, and in particular those sold by Dow Corning under the name DC 1248 or the oils Silwet© L 722, L 7500, L 77 and L 711 from Union Carbide; or also (C₁₂)alkyl methicone copolyols, and in particular those sold by Dow Corning under the name Q2-5200;
thiol groups, such as the products sold under the names GP 72 A and GP 71 from Genesee;
alkoxylated groups, such as the products sold under the names Silicone Copolymer F-755 by SWS Silicones and Abil Wax® 2428, 2434 and 2440 by Goldschmidt;
hydroxylated groups, such as polyorganosiloxanes having a hydroxyalkyl function;
acyloxyalkyl groups, such as the polyorganosiloxanes described in U.S. Pat. No. 4,957,732;
anionic groups of the carboxylic acid type, as described, for example, in EP 186 507, or of the alkylcarboxylic type, such as the product X-22-3701 E from Shin-Etsu;
or also of the 2-hydroxyalkylsulfonate or 2-hydroxyalkylthiosulfate type, such as the products sold by Goldschmidt under the names Abil© S201 and Abil© S255.

The silicones can also be chosen from polydialkylsiloxanes, among which mention may be made mainly of polydimethylsiloxanes having trimethylsilyl end groups (CTFA: dimethicone). Mention may be made, among these polydialkylsiloxanes, of the following commercial products:
the Silbione® oils of the 47 and 70 047 series or the Mirasil® oils sold by Rhodia, such as, for example, the oil 70 047 V 500 000;
the oils of the Mirasil® series sold by Rhodia;
the oils of the 200 series from Dow Corning, such as DC200 having a viscosity of 60 000 mm²/s;
the Viscasil® oils from General Electric and certain oils of the SF series (SF 96, SF 18) from General Electric.

Mention may also be made of polydimethylsiloxanes having dimethylsilanol end groups (CTFA: dimethiconol), such as the oils of the 48 series from Rhodia.

In this category of polydialkylsiloxanes, mention may also be made of the products sold under the names Abil Wax® 9800 and 9801 by Goldschmidt, which are polydi(C₁-C₂₀)alkylsiloxanes.

Products which can more particularly be used in accordance with the invention are mixtures such as:
mixtures formed from a polydimethylsiloxane hydroxylated at the chain end, or dimethiconol (CTFA), and from a cyclic polydimethylsiloxane, also known as cyclomethicone (CTFA), such as the product Q2-1401 sold by Dow Corning.

The polyalkylarylsiloxanes are particularly chosen from linear and/or branched polydimethyl/methylphenylsiloxanes or polydimethyl/diphenylsiloxanes with a viscosity ranging from 1.10-5 to 5.10⁻² m²/s at 25° C.

Mention may be made, among these polyalkylarylsiloxanes, of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 or SF 1265.

The non-aminated silicones more particularly preferred according to the invention are polydimethylsiloxanes having trimethylsilyl end groups (CTFA: dimethicone). The composition according to the invention can comprise one or more aminated silicones.

The term “aminated silicone” denotes any silicone comprising at least one primary, secondary or tertiary amine or a quaternary ammonium group.

The aminated silicones capable of being used according to the present invention can be volatile or non-volatile and cyclic, linear or branched, and preferably exhibit a viscosity ranging from 5×10⁻⁶ to 2.5 m²/s at 25° C., for example from 1×10⁻⁵ to 1 m²/s.

Preferably, the aminated silicone(s) are chosen, alone or as mixtures, from the following compounds:
A) the polysiloxanes corresponding to the formula (I):

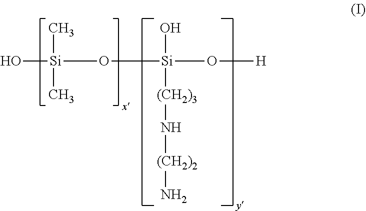

in which x′ and y′ are integers such that the weight-average molecular weight (Mw) is of between 5000 and 500 000 g/mol;
B) the aminated silicones corresponding to the formula 11):

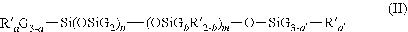

in which:
G, which is identical or different, denotes a hydrogen atom or a phenyl, OH, C₁-C₈ alkyl, for example methyl, or C₁-C₈ alkoxy, for example methoxy, group;
a and a′, which are identical or different, denote 0 or an integer from 1 to 3, in particular 0, with the proviso that at least one from a and a′ is equal to zero,
b denotes 0 or 1, in particular 1,
m and n are numbers such that the sum (n+m) varies from 1 to 2000 and in particular from 50 to 150, it being possible for n to denote a number from 0 to 1999 and in particular from 49 to 149 and it being possible for m to denote a number from 1 to 2000 and in particular from 1 to 10; and
R′, which is identical or different, denotes a monovalent radical of formula —C_{q}H_{2q}L in which q is a number ranging from 2 to 8 and L is an optionally quaternized amino group chosen from the following groups: —NR″-Q-N(R″)₂, —N(R″)₂, —N³⁰ (R″)₃ A⁻, —N⁺H(R″)₂ A⁻, —N⁺H₂(R″) A⁻, —NR″-Q-N³⁰ (R″)H₂ A⁻, —NR″-Q-N³⁰ (R″)₂H A⁻ and —NR″-Q-N³⁰ (R″)₃ A⁻, in which R″, which is identical or different, denotes hydrogen, phenyl, benzyl or a saturated monovalent hydrocarbon radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, in particular a halide, such as fluoride, chloride, bromide or iodide.

Preferably, the aminated silicones of formula (II) can be chosen from: (i) the “trimethylsilyl amodimethicone” silicones corresponding to the formula (III):

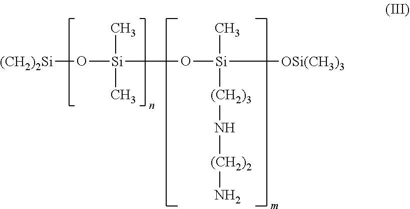

in which m and n are numbers such that the sum (n+m) varies from 1 to 2000, preferably from 20 to 1000, in particular from 50 to 600, better still from 50 to 150; it being possible for n to denote a number from 0 to 1999 and in particular from 49 to 149 and it being possible for m to denote a number from 1 to 2000 and in particular from 1 to 10;
(ii) the silicones of following formula (IV):

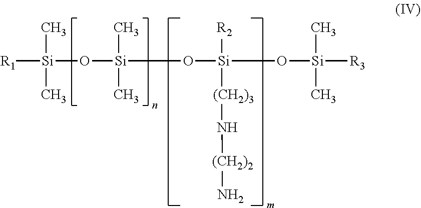

in which:
m and n are numbers such that the sum (n+m) varies from 1 to 1000, in particular from 50 to 250 and more particularly from 100 to 200; n denoting a number from 0 to 999 and in particular from 49 to 249 and more particularly from 125 to 175 and m denoting a number from 1 to 1000, in particular from 1 to 10 and more particularly from 1 to 5; and
R₁, R₂ and R₃, which are identical or different, represent a hydroxyl or C₁-C₄ alkoxy radical, at least one of the radicals R₁ to R₃ denoting an alkoxy radical.

Preferably, the alkoxy radical is a methoxy radical.

The hydroxy/alkoxy molar ratio preferably ranges from 0.2:1 to 0.4:1 and preferably from 0.25:1 to 0.35:1 and more particularly is equal to 0.3:1.

The weight-average molecular weight (Mw) of these silicones preferably ranges from 2000 to 1 000 000 g/mol and more particularly from 3500 to 200 000 g/mol.
(iii) the silicones of following formula (V):

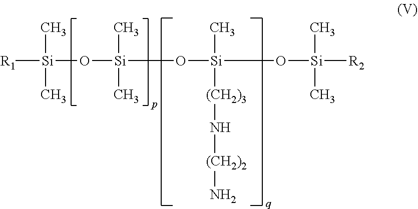

in which:
p and q are numbers such that the sum (p+q) varies from 1 to 1000, in particular from 50 to 350 and more particularly from 150 to 250; p denoting a number from 0 to 999, in particular from 49 to 349 and more particularly from 159 to 239 and q denoting a number from 1 to 1000, in particular from 1 to 10 and more particularly from 1 to 5; and
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy molar ratio generally ranges from 1:0.8 to 1:1.1 and preferably from 1:0.9 to 1:1 and more particularly is equal to 1:0.95.

The weight-average molecular weight (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) can include, in their composition, one or more other aminated silicones, the structure of which is different from the formula (IV) or (V). A product containing aminated silicones of structure (IV) is provided by Wacker under the name Belsil® ADM 652. A product containing aminated silicones of structure (V) is provided by Wacker under the name Fluid WR 1300®. Another product containing aminated silicones of structure (XIV) is provided by Wacker under the name Belsil ADM LOG 1®.

When these aminated silicones are employed, a particularly advantageous embodiment is their use in the form of an oil-in-water emulsion. The oil-in-water emulsion can comprise one or more surfactants. The surfactants can be of any nature but are preferably cationic and/or non-ionic. The number-average size of the silicone particles in the emulsion generally ranges from 3 nm to 500 nm. Preferably, in particular as aminated silicones of formula (V), use is made of microemulsions, the mean size of the particles of which ranges from 5 nm to 60 nm (limits included) and more particularly from 10 nm to 50 nm (limits included). Thus, use may be made, according to the invention, of the microemulsions of aminated silicone of formula (V) provided under the name Finish CT 96 E® or SLM 28020® by Wacker.
(iv) the silicones of following formula (VI):

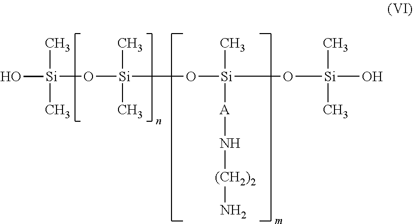

in which:
m and n are numbers such that the sum (n+m) varies from 1 to 2000 and in particular from 50 to 150, n denoting a number from 0 to 1999 and in particular from 49 to 149 and m denoting a number from 1 to 2000 and in particular from 1 to 10; and
A denotes a linear or branched alkylene radical having from 4 to 8 carbon atoms and preferably 4 carbon atoms. This radical is preferably linear.

The weight-average molecular weight (Mw) of these aminated silicones preferably ranges from 2000 to 1 000 000 g/mol and more particularly from 3500 to 200 000 g/mol.

A silicone corresponding to this formula is, for example, Xiameter MEM 8299 Emulsion from Dow Corning.
(v) the silicones of following formula (VII):

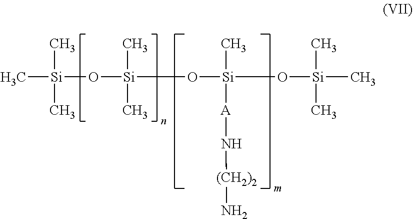

in which:
m and n are numbers such that the sum (n+m) varies from 1 to 2000 and in particular from 50 to 150, it being possible for n to denote a number from 0 to 1999 and in particular from 49 to 149 and it being possible for m to denote a number from 1 to 2000 and in particular from 1 to 10; and
A denotes a linear or branched alkylene radical having from 4 to 8 carbon atoms and preferably 4 carbon atoms. This radical is preferably branched.

The weight-average molecular weight (Mw) of these aminated silicones preferably ranges from 500 to 1 000 000 g/mol and more particularly from 1000 to 200 000 g/mol.

A silicone corresponding to this formula is, for example, DC2-8566 Amino Fluid from Dow Corning;
C) the aminated silicones corresponding to formula (VIII):

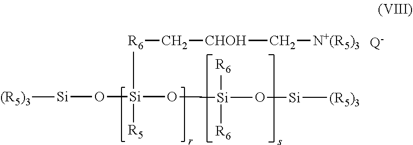

in which:
R₅ represents a monovalent hydrocarbon radical having from 1 to 18 carbon atoms and in particular a C₁-C₁₈ alkyl or C₂-C₁₈ alkenyl radical, for example a methyl radical;
R₆ represents a divalent hydrocarbon radical, in particular a C₁-C₁₈ alkylene radical, or a divalent C₁-C₁₈, for example C₁-C₈, alkyleneoxy radical connected to the Si via an SiC bond;
Q⁻ is an anion, such as a halide ion, in particular a chloride ion, or an organic acid salt, in particular acetate;
r represents a mean statistical value ranging from 2 to 20, in particular from 2 to 8;
and
s represents a mean statistical value ranging from 20 to 200, in particular from 20 to 50;
D) the silicones having quaternary ammonium of formula (IX):

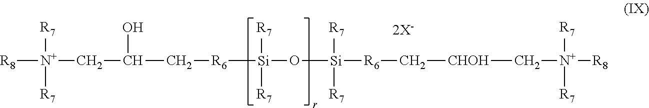

in which:
R₇, which are identical or different, represent a monovalent hydrocarbon radical having from 1 to 18 carbon atoms and in particular a C₁-C₁₈ alkyl radical, a C₂-C₁₈ alkenyl radical or a ring comprising 5 or 6 carbon atoms, for example a methyl radical;
R₆ represents a divalent hydrocarbon radical, in particular a C₁-C₁₈ alkylene radical, or a divalent C₁-C₁₈, for example C₁-C₈, alkyleneoxy radical connected to the Si via an SiC bond;
R₅, which are identical or different, represent a hydrogen atom, a monovalent hydrocarbon radical having from 1 to 18 carbon atoms, in particular a C₁-C₁₈ alkyl radical or a C₂-C₁₈ alkenyl radical, or a —R₆—NHCOR₇ radical;
X⁻ is an anion, such as a halide ion, in particular a chloride ion, or an organic acid salt, in particular acetate; and
r represents a mean statistical value ranging from 2 to 200, in particular from 5 to 100.

These silicones are for example described in Application EP-A-0 530 974; mention may in particular be made of the silicone with the INCI name: Quaternium 80. Silicones coming within this category are the silicones sold by Goldschmidt under the names Abil Quat 3270, Abil Quat 3272 and Abil Quat 3474;
E) the aminated silicones of formula (X):

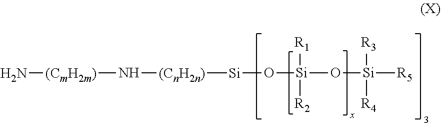

in which:
R₁, R₂, R₃ and R₄, which are identical or different, denote a C₁-C₄ alkyl radical or a phenyl group,
R₅ denotes a C₁-C₄ alkyl radical or a hydroxyl group,
n is an integer varying from 1 to 5,
m is an integer varying from 1 to 5, and
x is chosen such that the amine number varies from 0.01 to 1 meq/g;
F) the multiblock polyoxyalkylenated aminated silicones, of (AB)ₙ type, A being a polysiloxane block and B being a polyoxyalkylenated block comprising at least one amine group.

Said silicones are preferably constituted of repeat units with the following general formulae:

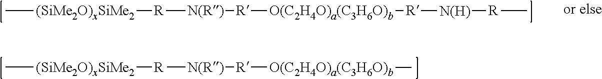

in which:
a is an integer greater than or equal to 1, preferably ranging from 5 to 200 and more particularly ranging from 10 to 100;
b is an integer of between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which are identical or different, represent a linear or branched divalent C₂-C₁₂ hydrocarbon radical, optionally comprising one or more heteroatoms, such as oxygen; preferably, R, which are identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which are identical or different, represent a linear or branched divalent C₂-C₁₂ hydrocarbon radical, optionally comprising one or more heteroatoms, such as oxygen; preferably, R′, which are identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.

The siloxane blocks preferably represent between 50 mol % and 95 mol % of the total weight of the silicone, more particularly from 70 mol % to 85 mol %.

The amine content is preferably of between 0.02 and 0.5 meq/g of copolymer in a 30% solution in dipropylene glycol, more particularly between 0.05 and 0.2.

The weight-average molecular weight (Mw) of the silicone is preferably of between 5000 and 1 000 000 g/mol and more particularly between 10 000 and 200 000 g/mol.

Mention may in particular be made of the silicones sold under the name Silsoft A-843 or Silsoft A+ by Momentive;
G) the aminated silicones of formulae (XI) and (XII):

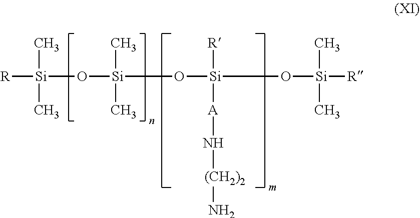

in which:
R, R′ and R″, which are identical or different, denote a C₁-C₄ alkyl group or a hydroxyl group,
A denotes a C₃ alkylene radical; and
m and n are numbers such that the weight-average molecular weight of the compound is of between 5000 and 500 000;

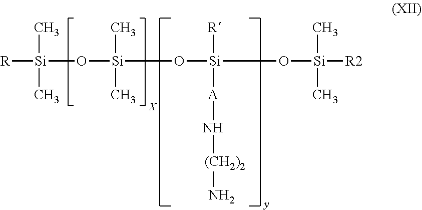

in which:
x and y are numbers ranging from 1 to 5000; preferably, x ranges from 10 to 2000 and more preferentially from 100 to 1000; preferably, y ranges from 1 to 100;
R₁ and R₂, which are identical or different, preferably identical, denote a saturated or unsaturated, linear or branched, alkyl group comprising from 6 to 30 carbon atoms, preferably from 8 to 24 carbon atoms and more preferentially from 12 to 20 carbon atoms; and
A denotes a linear or branched alkylene radical having from 2 to 8 carbon atoms.

Preferably, A comprises from 3 to 6 carbon atoms, more preferentially 4 carbon atoms; preferably, A is branched. Mention may be made in particular of the following divalent groups: —CH₂CH₂CH₂— and —CH₂CH(CH₃)CH₂—.

Preferably, R₁ and R₂ are independent saturated linear alkyl groups comprising from 6 to 30 carbon atoms, preferably from 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which are identical or different, are chosen from hexadecyl (cetyl) and octadecyl (stearyl) groups.

Preferably, in the silicone of formula (XII):
x ranges from 10 to 2000 and in particular from 100 to 1000;
y ranges from 1 to 100;
A comprises from 3 to 6 carbon atoms and in particular 4 carbon atoms; preferably,
A is branched; more particularly, A is chosen from the following divalent groups: —CH₂CH₂CH₂ and —CH₂CH(CH₃)CH₂—; and
R₁ and R₂ are independently saturated linear alkyl groups comprising from 6 to 30 carbon atoms, preferably from 8 to 24 carbon atoms and in particular from 12 to 20 carbon atoms; chosen in particular from dodecyl, tetradecyl, pentadecyl, hexadecyl, heptadecyl, octadecyl, nonadecyl and eicosyl groups; preferentially, R₁ and R₂, which are identical or different, are chosen from hexadecyl (cetyl) and octadecyl (stearyl) groups.

A preferred silicone of formula (XII) is bis-cetearyl amodimethicone. Mention may in particular be made of the aminated silicone sold under the name Silsoft AX by Momentive;
H) the polysiloxanes and in particular the polydimethylsiloxanes comprising primary amine groups at only one chain end or on side chains, such as those of formula (XIV), (XV) or (XVI):

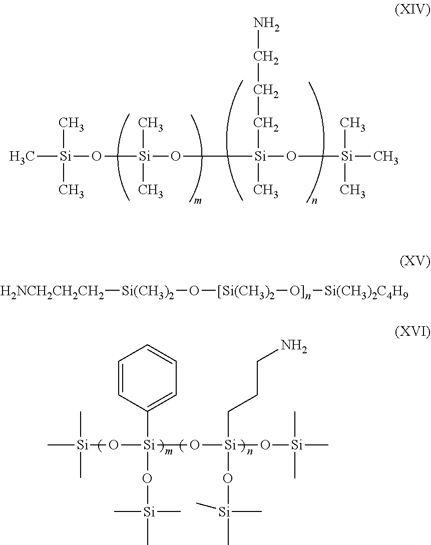

In the formula (XIV), the values of n and m are such that the weight-average molecular weight of the aminated silicone is of between 1000 and 55 000.

Mention may be made, as examples of aminated silicones of formula (XIV), of the products sold under the names AMS-132, AMS-152, AMS-162, AMS-163, AMS-191 and AMS-1203 by Gelest and KF-8015 by Shin-Etsu.

In the formula (XV), the value of n is such that the weight-average molecular weight of the aminated silicone is of between 500 and 3000.

Mention may be made, as examples of aminated silicones of formula (XV), of the products sold under the names MCR-A11 and MCR-A12 by Gelest.

In the formula (XVI), the values of n and m are such that the weight-average molecular weight of the aminated silicone is of between 500 and 50 000.

Mention may be made, as examples of aminated silicones of formula (XVI), of the aminopropyl phenyl trimethicone sold under the name DC 2-2078 Fluid by Dow Corning.

The cosmetic composition according to the invention can also comprise, as silicone, an aminated silicone corresponding to the formula (XVIII) below:

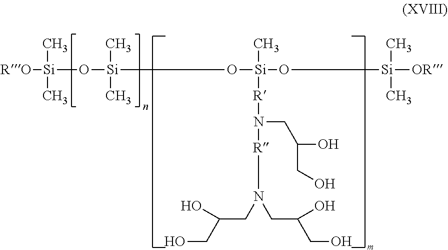

in which:
n is a number of between 1 and 1000, preferably between 10 and 500, better still between 25 and 100, even better still between 50 and 80;
m is a number of between 1 and 200, preferably between 1 and 100, better still between 1 and 10 and even better still between 1 and 5;
R″′, which are identical or different, preferably identical, are saturated or unsaturated, linear or branched, alkyl radicals comprising from 8 to 30 carbon atoms, preferably from 10 to 24 carbon atoms, in particular from 12 to 18 carbon atoms; it being possible for said radicals optionally to be substituted with one or more hydroxyl OH groups;
R′ is a linear or branched divalent alkylene radical having from 1 to 6 carbon atoms, in particular from 2 to 5 carbon atoms;
R″ is a linear or branched divalent alkylene radical having from 1 to 6 carbon atoms, in particular from 1 to 5 carbon atoms.

Preferably, the R″′ radicals, which are identical or different, are saturated linear alkyl radicals comprising from 8 to 30 carbon atoms, preferably from 10 to 24 carbon atoms, in particular from 12 to 18 carbon atoms; mention may be made in particular of dodecyl, tridecyl, tetradecyl, pentadecyl, hexadecyl, heptadecyl and octadecyl radicals; preferentially, the R″′ radicals, which are identical or different, are chosen from saturated linear alkyl radicals having from 12 to 16 carbon atoms, in particular which are C₁₃, C₁₄ or C₁₅ radicals, alone or as a mixture, and better still represent a mixture of C₁₃, C₁₄ and C₁₅ radicals.

Preferably, the R″′ radicals are identical.

Preferably, R′ is a linear or branched, preferably branched, divalent alkylene radical comprising from 1 to 6 carbon atoms, in particular from 2 to 5 carbon atoms; in particular a —CH₂—CH₂—CH₂—, —CH₂—CH(CH₃)—CH₂— or —CH₂—CH₂—CH(CH₃)— radical. Preferably, R″ is a linear divalent alkylene radical comprising from 1 to 6 carbon atoms, in particular from 1 to 4 carbon atoms, especially a —CH₂—CH₂— radical.

Preferentially, the composition can comprise one or more silicones of formula (XVIII)

in which:
n is a number of between 50 and 80;
m is a number of between 1 and 5;
R″′, which are identical, are saturated linear alkyl radicals comprising from 12 to 18 carbon atoms;
R′ is a divalent alkylene radical having from 2 to 5 carbon atoms;
R″ is a linear divalent alkylene radical having from 1 to 4 carbon atoms.

Better still, the composition can comprise one or more silicones of formula (XVIII) in which:
n is a number of between 50 and 80;
m is a number of between 1 and 5;
R″′, which are identical, are saturated linear alkyl radicals comprising from 13 to 15 carbon atoms;
R′ is a —(CH₂)₃—, —CH₂—CH(CH₃)—CH₂— or —CH₂—CH₂—CH(CH₃)— radical, and
R″ is a —(CH₂)₂— radical.

A very particularly preferred silicone of formula (XVIII) is Bis(C₁₃₋₁₅ Alkoxy) PG-Amodimethicone (INCI name). Mention may in particular be made of the silicone sold under the name Dowsil 8500 Conditioning Agent by Dow.

Preferably, the aminated silicone(s) are chosen, alone or as a mixture, from:
the aminated silicones of formula (II), preferably those in which G denotes a C₁-C₈ alkyl and a=a′=0; a very particularly preferred silicone of formula (II) being aminopropyl dimethicone, such as that sold under the name X-22-9686 by Shin-Etsu;
the silicones having quaternary ammonium of formula (IX) above;
the aminated silicones of formula (XVIII) above, in particular those in which n is a number of between 50 and 80; m is a number of between 1 and 5; R″′, which are identical, are saturated linear alkyl radicals comprising from 13 to 15 carbon atoms; R′ is a —(CH₂)₃—, —CH₂—CH(CH₃)—CH₂— or —CH₂—CH₂—CH(CH₃)— radical, and R″ is a —(CH₂)₂— radical.

Advantageously, the composition according to the present invention can comprise the silicone(s) in a total content preferably ranging from 0.3% to 5% by weight, better still from 0.5% to 4% by weight, preferentially from 0.6% to 3% by weight and more preferentially from 0.7% to 2.5% by weight, with respect to the total weight of the composition.

Advantageously, the composition according to the present invention can comprise the aminated and non-aminated silicone(s) in a total content preferably ranging from 0.3% to 5% by weight, better still from 0.5% to 4% by weight, preferentially from 0.6% to 3% by weight and even better still from 0.7% to 2.5% by weight, with respect to the total weight of the composition.

### Cationic Surfactants

The composition used according to the invention can optionally comprise one or more cationic surfactants.

Said cationic surfactants are non-silicone surfactants, that is to say that they do not contain an Si—O group.

They are preferably chosen from quaternary ammonium salts, primary, secondary or tertiary fatty amines, which are optionally polyoxyalkylenated, or their salts, and their mixtures.

The composition can comprise one or more cationic surfactants chosen, alone or as a mixture, from the following compounds, which are quaternary ammonium salts:
the compounds corresponding to the following general formula (II):

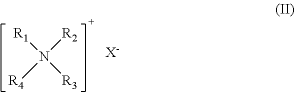

in which:
X⁻ is an anion in particular chosen from the group of halides, phosphates, acetates, lactates, (C₁-C₄)alkyl sulfates, (C₁-C₄)alkylsulfonates or (C₁-C₄)alkylarylsulfonates, the groups R₁ to R₄, which can be identical or different, represent a linear or branched aliphatic group comprising from 1 to 30 carbon atoms, or an aromatic group, such as aryl or alkylaryl, at least one of the groups R₁ to R₄ denoting a linear or branched aliphatic group comprising from 8 to 30 carbon atoms, preferably from 12 to 24 carbon atoms.

The aliphatic groups can comprise heteroatoms, such as in particular oxygen, nitrogen, sulfur and halogens. The aliphatic groups are, for example, chosen from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups.

Preference is given, among the quaternary ammonium salts of formula (II), to tetraalkylammonium salts, such as dialkyldimethylammonium or alkyltrimethylammonium salts, in which the alkyl group comprises approximately from 12 to 22 carbon atoms, in particular behenyltrimethylammonium, distearyldimethylammonium, cetyltrimethylammonium or benzyldimethylstearylammonium salts, and also palmitylamidopropyltrimethylammonium salts, stearamidopropyltrimethylammonium salts, stearamidopropyldimethylcetearylammonium salts or stearamidopropyldimethyl(myristyl acetate)ammonium salts, such as those sold under the name Ceraphyl® 70 by Van Dyk.

It is preferred in particular to use the chloride, bromide or methyl sulfate salts of these compounds.
the quaternary ammonium salts of imidazoline, such as those of formula (III):

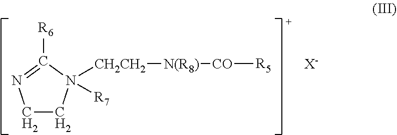

in which 1 represents an alkenyl or alkyl group comprising from 8 to 30 carbon atoms, for example derived from tallow fatty acids, R₆ represents a hydrogen atom, a C₁-C₄ alkyl group or an alkyl or alkenyl group comprising from 8 to 30 carbon atoms, R₇ represents a C₁-C₄ alkyl group, R₈ represents a hydrogen atom or a C₁-C₄ alkyl group, X⁻ is an anion chosen from the group of halides, phosphates, acetates, lactates, alkyl sulfates, alkyl- or alkylarylsulfonates, the alkyl and aryl groups of which preferably comprise, respectively, from 1 to 20 carbon atoms and from 6 to 30 carbon atoms.

Preferably, R₅ and R₆ denote a mixture of alkenyl or alkyl groups comprising from 12 to 21 carbon atoms, for example derived from tallow fatty acids, R₇ denotes a methyl group and R₈ denotes a hydrogen atom.

Such a product is sold, for example, under the name Rewoquat® W 75 by Rewo;
di- or triquaternary ammonium salts, in particular of formula (IV):

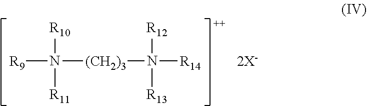

in which:
R₉ denotes an alkyl radical comprising approximately from 16 to 30 carbon atoms which is optionally hydroxylated and/or optionally interrupted by one or more oxygen atoms,
R₁₀ is chosen from hydrogen or an alkyl radical comprising from 1 to 4 carbon atoms or a (R₉ₐ)(R₁₀ₐ)(R₁₁ₐ)N—(CH₂)₃ group, with R₉ₐ, R₁₀ₐ, R₁₁ₐ, R₁₁, R₁₂, R₁₃ and R₁₄, which are identical or different, chosen from hydrogen or an alkyl radical comprising from 1 to 4 carbon atoms, and
X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, provided by Finetex (Quaternium 89), and Finquat CT, provided by Finetex (Quaternium 75);
quaternary ammonium salts containing at least one ester function, such as those of following formula (V):

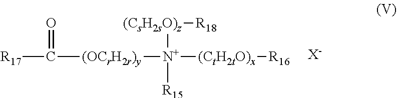

in which:
R₁₅ is chosen from C₁-C₆ alkyl groups and C₁-C₆ hydroxyalkyl or dihydroxyalkyl groups;
R₁₆ is chosen from the R₁₉—C(O)— group; R₂₀ groups which are saturated or unsaturated, linear or branched, C₁-C₂₂ hydrocarbon groups; a hydrogen atom;
R₁₈ is chosen from the R₂₁—C(O)— group; R₂₂ groups which are saturated or unsaturated, linear or branched, C₁-C₆ hydrocarbon groups; a hydrogen atom;
R₁₇, R₁₉ and R₂₁, which are identical or different, are chosen from saturated or unsaturated, linear or branched, C₇-C₂₁ hydrocarbon groups;
r, s and t, which are identical or different, are integers having values from 2 to 6; y is an integer having a value from 1 to 10;
x and z, which are identical or different, are integers having a value from 0 to 10;
X⁻ is a simple or complex, organic or inorganic, anion;
with the proviso that the sum x+y+z has a value from 1 to 15, that, when x has the value 0, then R₁₆ denotes R₂₀, and that, when z has the value 0, then R₁₈ denotes R₂₂.

The alkyl groups R₁₅ can be linear or branched and more particularly linear. Preferably, R₁₅ denotes a methyl, ethyl, hydroxyethyl or dihydroxypropyl group and more particularly a methyl or ethyl group.

Advantageously, the sum x+y+z has a value from 1 to 10.

When R₁₆ is a hydrocarbon group R₂₀, it can be long and have from 12 to 22 carbon atoms or short and have from 1 to 3 carbon atoms.

When R₁₈ is a hydrocarbon group R₂₂, it preferably has from 1 to 3 carbon atoms. Advantageously, R₁₇, R₁₉ and R₂₁, which are identical or different, are chosen from saturated or unsaturated, linear or branched, C₁₁-C₂₁ hydrocarbon groups and more particularly from saturated or unsaturated, linear or branched, C₁₁-C₂₁ alkyl and alkenyl groups.

Preferably, x and z, which are identical or different, have the value 0 or 1.

Advantageously, y is equal to 1.

Preferably, r, s and t, which are identical or different, have the value 2 or 3 and more particularly still are equal to 2.

The anion X⁻ is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion compatible with the ammonium having an ester function. The anion X⁻ is more particularly still chloride or methyl sulfate.

Use may more particularly be made, in the composition according to the invention, of the ammonium salts of formula (V) in which R₁₅ denotes a methyl or ethyl group, x and y are equal to 1, z is equal to 0 or 1 and r, s and t are equal to 2;
R₁₆ is chosen from the R₁₉—C(O)— group, methyl, ethyl or C₁₄-C₂₂ hydrocarbon groups, and a hydrogen atom;
R₁₈ is chosen from the R₂₁—C(O)— group and a hydrogen atom;
R₁₇, R₁₉ and R₂₁, which are identical or different, are chosen from saturated or unsaturated, linear or branched, C₁₃-C₁₇ hydrocarbon groups and preferably from saturated or unsaturated, linear or branched, C₁₃-C₁₇ alkyl and alkenyl groups.

Advantageously, the hydrocarbon groups are linear.

Mention may be made, for example, of the compounds of formula (V), such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular) and their mixtures. The acyl groups preferably have from 14 to 18 carbon atoms and originate more particularly from a vegetable oil, such as palm oil or sunflower oil. When the compound contains several acyl groups, the latter can be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of vegetable or animal origin, or by transesterification of their methyl esters. This esterification is followed by a quaternization using an alkylating agent, such as an alkyl (preferably methyl or ethyl) halide, a dialkyl (preferably dimethyl or diethyl) sulfate, methyl methanesulfonate, methyl para-toluenesulfonate, glycol chlorohydrin or glycerol chlorohydrin.

Such compounds are sold, for example, under the names Dehyquart® by Henkel, Stepanquat® by Stepan, Noxamium® by CECA or Rewoquat® WE 18 by Rewo-Witco.

The composition according to the invention can contain, for example, a mixture of quaternary ammonium mono-, di- and triester salts with a majority by weight of diester salts.

Use may also be made of the ammonium salts containing at least one ester function which are described in Patents U.S. Pat. Nos. 4,874,554 and 4,137,180.

Use may be made of behenoylhydroxypropyltrimethylammonium chloride provided by Kao under the name Quatarmin BTC 131.

Preferably, the ammonium salts containing at least one ester function contain two ester functions.

It is preferable, among the quaternary ammonium salts containing at least one ester function which can be used, to use dipalmitoylethylhydroxyethylmethylammonium salts.

“Fatty amine” is understood to mean a compound comprising at least one primary, secondary or tertiary amine function, which is optionally (poly)oxyalkylenated, or their salts and comprising at least one C₆-C₃₀, preferably C₈-C₃₀, hydrocarbon chain.

Preferably, the fatty amines of use according to the invention are not (poly)oxyalkylenated.

Mention may be made, by way of fatty amines, of amidoamines. The amidoamines according to the invention can be chosen from fatty amidoamines, it being possible for the fatty chain to be carried by the amine group or by the amido group.

“Amidoamine” is understood to mean a compound comprising at least one amide function and at least one primary, secondary or tertiary amine function.

“Fatty amidoamine” is understood to mean an amidoamine comprising, in general, at least one C₆-C₃₀ hydrocarbon chain. Preferably, the fatty amidoamines of use according to the invention are not quaternized.

Preferably, the fatty amidoamines of use according to the invention are not (poly)oxyalkylenated.

Mention may be made, among the fatty amidoamines of use according to the invention, of the amidoamines of following formula (VI): RCONHR″N(R′)₂ (VI) in which:
R represents a substituted or unsubstituted, saturated or unsaturated, linear or branched, monovalent hydrocarbon radical having from 5 to 29 carbon atoms, preferably from 7 to 23 carbon atoms, and in particular a linear or branched C₅-C₂₉, preferably C₇-C₂₃, alkyl radical or a linear or branched C₅-C₂₉, preferably C₇-C₂₃, alkenyl radical;
R″ represents a divalent hydrocarbon radical having less than 6 carbon atoms, preferably from 2 to 4 carbon atoms and better still 3 carbon atoms; and
R′, which are identical or different, represent a substituted or unsubstituted, saturated or unsaturated, linear or branched, monovalent hydrocarbon radical having less than 6 carbon atoms, preferably from 1 to 4 carbon atoms, preferably a methyl radical.

The fatty amidoamines of formula (VI) are chosen, for example, from oleamidopropyldimethylamine, stearamidopropyldimethylamine, in particular the product sold by Inolex Chemical Company under the name Lexamine S13, isostearamidopropyldimethylamine, stearamidoethyldimethylamine, lauramidopropyldimethylamine, myristamidopropyldimethylamine, behenamidopropyldimethylamine, dilinoleamidopropyldimethylamine, palmitamidopropyldimethylamine, ricinoleamidopropyldimethylamine, soyamidopropyldimethylamine, avocadoamidopropyldimethylamine, cocamidopropyldimethylamine, minkamidopropyldimethylamine, oatamidopropyldimethylamine, sesamidopropyldimethylamine, tallamidopropyldimethylamine, olivamidopropyldimethylamine, palm itamidopropyldimethylamine, stearamidoethyldiethylamine, brassicamidopropyldimethylamine and their mixtures.

Preferably, the fatty amidoamines are chosen from oleamidopropyldimethylamine, stearamidopropyldimethylamine, brassicamidopropyldimethylamine and their mixtures.

The cationic surfactant(s) are preferably chosen from those of formula (II) above, those of formula (V) above, those of formula (VI) above and their mixtures; better still from those of formula (II) above, those of formula (VI) above and their mixtures; even better still from those of formula (II) above.

Preferentially, the cationic surfactant(s) can be chosen from tetraalkylammonium salts, such as chlorides, bromides or methosulfates, such as, for example, dialkyldimethylammonium or alkyltrimethylammonium salts in which the alkyl group comprises approximately from 12 to 22 carbon atoms, in particular behenyltrimethylammonium, distearyldimethylammonium, cetyltrimethylammonium or benzyldimethylstearylammonium salts; dipalmitoylethylhydroxyethylmethylammonium salts, such as dipalmitoylethylhydroxyethylmethylammonium methosulfate and their mixtures.

More preferentially still, they are chosen from cetyltrimethylammonium chloride, behenyltrimethylammonium chloride, dipalmitoylethylhydroxyethylmethylammonium methosulfate and their mixtures.

When they are present, the total content of cationic surfactant(s) in the composition according to the invention preferably ranges from 0.1% to 10% by weight, in particular from 0.2% to 8% by weight, better still from 0.3% to 7% by weight, even better still from 0.5% to 5% by weight, with respect to the total weight of the composition.

In a preferred embodiment of the invention, the ratio by weight of the total content of cationic surfactant(s) to the total content of compounds of amino acids type is greater than or equal to 1.

### Cationic Polymers

The composition used according to the invention can optionally comprise one or more cationic polymers other than the associative polymers described above. This or these cationic polymer(s) are thus non-associative.

“Cationic polymer” is understood to mean any non-silicone polymer (polymer not comprising a silicon atom) containing cationic groups and/or groups which can be ionized to give cationic groups and not containing anionic groups and/or groups which can be ionized to give anionic groups.

The cationic polymers capable of being employed preferably have a cationic charge density of less than or equal to 5 milliequivalents/gram (meq/g), better still of less than or equal to 4 meq/g.

The cationic charge density of a polymer corresponds to the number of moles of cationic charges per unit weight of polymer under the conditions where the latter is completely ionized. It can be determined by calculation if the structure of the polymer is known, that is to say the structure of the monomers constituting the polymer and their proportion in moles or by weight. It can also be determined experimentally by the Kjeldahl method.

The cationic polymers capable of being used preferably have a weight-average molar mass (Mw) of between 500 and 5.10⁶ approximately, preferably of between 10³ and 3.10⁶ approximately.

The cationic polymers capable of being employed are preferably non-associative.

Mention may be made, among the cationic polymers capable of being employed, of: (1) homopolymers or copolymers derived from acrylic or methacrylic esters or amides and comprising at least one of the units with the following formulae:

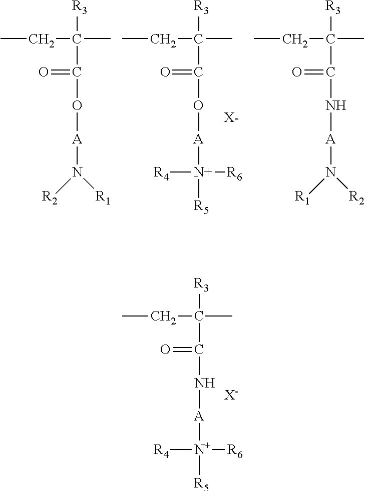

in which:
   R₃, which are identical or different, denote a hydrogen atom or a CH₃ radical;
   A, which are identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which are identical or different, represent an alkyl group having from 1 to 18 carbon atoms or a benzyl radical, preferably an alkyl group having from 1 to 6 carbon atoms;
   R₁ and R₂, which are identical or different, represent a hydrogen atom or an alkyl group having from 1 to 6 carbon atoms, preferably methyl or ethyl;
   X denotes an anion derived from a mineral or organic acid, such as a methosulfate anion or a halide, such as chloride or bromide.

The copolymers of family (1) can additionally contain one or more units deriving from comonomers which can be chosen from the family of acrylamides, methacrylamides, diacetone acrylamides, acrylamides and methacrylamides substituted on the nitrogen with lower (C₁-C₄) alkyls, acrylic or methacrylic acid esters, vinyllactams, such as vinylpyrrolidone or vinylcaprolactam, and vinyl esters.

Mention may be made, among these copolymers of the family (1), of:
copolymers of acrylamide and of dimethylaminoethyl methacrylate quaternized with dimethyl sulfate or with a dimethyl halide, such as that sold under the name Hercofloc by Hercules,
copolymers of acrylamide and of methacryloyloxyethyltrimethylammonium chloride, such as those sold under the name Bina Quat P 100 by Ciba-Geigy,
the copolymer of acrylamide and of methacryloyloxyethyltrimethylammonium methosulfate, such as that sold under the name Reten by Hercules,
quaternized or non-quaternized vinylpyrrolidone/dialkylaminoalkyl acrylate or methacrylate copolymers, such as the products sold under the name Gafquat by ISP, such as, for example, Gafquat 734 or Gafquat 755, or else the products known as Copolymer 845, 958 and 937,
dimethylaminoethyl methacrylate/vinylcaprolactam/vinylpyrrolidone terpolymers, such as the product sold under the name Gaffix VC 713 by ISP,
vinylpyrrolidone/methacrylamidopropyldimethylamine copolymers, such as those sold under the name Styleze CC 10 by ISP,
quaternized vinylpyrrolidone/dimethylaminopropylmethacrylamide copolymers, such as the product sold under the name Gafquat HS 100 by ISP,
polymers, preferably crosslinked, of methacryloyloxy(C₁-C₄)alkyltri(C₁-C₄)alkylammonium salts, such as the polymers obtained by homopolymerization of dimethylaminoethyl methacrylate quaternized with methyl chloride or by copolymerization of acrylamide with dimethylaminoethyl methacrylate quaternized with methyl chloride, the homopolymerization or the copolymerization being followed by crosslinking with an olefinically unsaturated compound, in particular methylenebisacrylamide. Use may more particularly be made of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by Ciba;
(2) cationic polysaccharides, in particular cationic celluloses and galactomannan gums.

Mention may more particularly be made, among the cationic polysaccharides, of cellulose ether derivatives comprising quaternary ammonium groups, cationic cellulose copolymers, cellulose derivatives grafted with a water-soluble quaternary ammonium monomer and cationic galactomannan gums.

The cellulose ether derivatives comprising quaternary ammonium groups are in particular described in FR 1 492 597; they are also defined in the CTFA dictionary as quaternary ammoniums of hydroxyethylcellulose which has reacted with an epoxide substituted with a trimethylammonium group.

Mention may in particular be made of the polymers sold under the name Ucare Polymer JR (JR 400 LT, JR 125 and JR 30M) or LR (LR 400 and LR 30M) by Amerchol.

Cationic cellulose copolymers and cellulose derivatives grafted with a water-soluble quaternary ammonium monomer are described in particular in Patent U.S. Pat. No. 4,131,576; mention may be made of hydroxyalkylcelluloses, such as hydroxymethyl-, hydroxyethyl- or hydroxypropylcelluloses grafted in particular with a methacryloylethyltrimethylammonium, methacrylamidopropyltrimethylammonium or dimethyldiallylammonium salt. Mention may very particularly be made of crosslinked or non-crosslinked quaternized hydroxyethylcelluloses, it being possible for the quaternizing agent to be in particular diallyldimethylammonium chloride; and very particularly hydroxyethylcellulose hydroxypropyltrimethylammonium.

Mention may be made, among the commercial products corresponding to this definition, of the products sold under the names Celquat L 200 and Celquat H 100 by National Starch.

Mention may in particular be made, as particularly preferred cationic cellulose, of the polymer with the INCI name Polyquaternium-10.

The cationic galactomannan gums are described in particular in Patents U.S. Pat. Nos. 3,589,578 and 4,031,307; mention may be made of cationic guar gums, in particular those comprising cationic trialkylammonium groups, in particular trimethylammonium groups. Mention may thus be made of guar gums modified with a 2,3-epoxypropyltrimethylammonium salt (for example a chloride).

Preferably, from 2% to 30% by number of the hydroxyl functions of the guar gums carry cationic trialkylammonium groups. More preferentially still, from 5% to 20% of the number of the hydroxyl functions of these guar gums are branched with cationic trialkylammonium groups. Mention may very particularly be made, among these trialkylammonium groups, of the trimethylammonium and triethylammonium groups. More preferentially still, these groups represent from 5% to 20% by weight, with respect to the total weight of the modified guar gum. According to the invention, guar gums modified with 2,3-epoxypropyltrimethylammonium chloride can be used.

Mention may in particular be made of the products with the INCI names Hydroxypropyl Guar Hydroxypropyltrimonium Chloride and Guar Hydroxypropyltrimonium Chloride. Such products are sold in particular under the names Jaguar C13S, Jaguar C15, Jaguar C17 and Jaguar C162 by Solvay.

Mention may also be made, among the cationic polysaccharides capable of being employed, of cationic derivatives of cassia gum, in particular those comprising quaternary ammonium groups; mention may especially be made of the product with the INCI name Cassia Hydroxypropyltrimonium Chloride;
(3) polymers constituted of piperazinyl units and of divalent alkylene or hydroxyalkylene radicals having linear or branched chains, optionally interrupted by oxygen, sulfur or nitrogen atoms or by aromatic or heterocyclic rings, and also the oxidation and/or quaternization products of these polymers;
(4) water-soluble polyaminoamides prepared in particular by polycondensation of an acid compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or also with an oligomer resulting from the reaction of a bifunctional compound which is reactive with regard to a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, quaternized;
(5) polyaminoamide derivatives resulting from the condensation of polyalkylenepolyamines with polycarboxylic acids, followed by alkylation with bifunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Mention may more particularly be made, among these derivatives, of the adipic acid/dimethylaminohydroxypropyl/diethylenetriamine polymers sold under the name Cartaretine F, F4 or F8 by Sandoz;
(6) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms; the molar ratio of the polyalkylenepolyamine to the dicarboxylic acid preferably being of between 0.8:1 and 1.4:1; the polyaminoamide resulting therefrom being reacted with epichlorohydrin in a molar ratio of epichlorohydrin, with respect to the secondary amine group of the polyaminoamide, of preferably between 0.5:1 and 1.8:1. Polymers of this type are sold in particular under the name Hercosett 57 by Hercules Inc. or else under the name PD 170 or Delsette 101 by Hercules in the case of the adipic acid/epoxypropyl/diethylenetriamine copolymer.
(7) cyclopolymers of alkyldiallylamine or of dialkyldiallylammonium, such as the homopolymers or copolymers comprising, as main constituent of the chain, units corresponding to the formula (I) or (11):

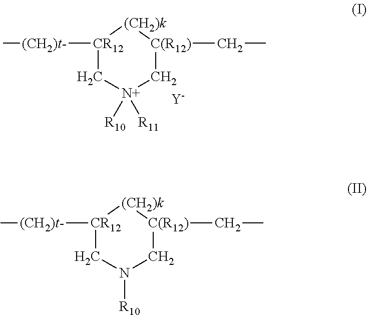

in which:
k and t are equal to 0 or 1, the sum k+t being equal to 1;
R₁₂ denotes a hydrogen atom or a methyl radical;
R₁₀ and R₁₁, independently of each other, denote a C₁-C₆ alkyl group, a C₁-C₅ hydroxyalkyl group, a C₁-C₄ amidoalkyl group; or else R₁₀ and R₁₁ can denote, together with the nitrogen atom to which they are attached, a heterocyclic group, such as piperidinyl or morpholinyl; R₁₀ and R₁₁, independently of each other, preferably denote a C₁-C₄ alkyl group;
Y⁻ is an anion, such as bromide, chloride, acetate, borate, citrate, tartrate, bisulfate, bisulfite, sulfate or phosphate.

Mention may more particularly be made of the homopolymer of dimethyldiallylammonium salts (for example chloride) (INCI name: Polyquaternium-6), for example sold under the name Merquat 100 by Nalco, and the copolymers of diallyldimethylammonium salts (for example chloride) and of acrylamide (INCI name: Polyquaternium-7), sold in particular under the name Merquat 550 or Merquat 7SPR;
(8) diquaternary ammonium polymers comprising repeat units of formula:

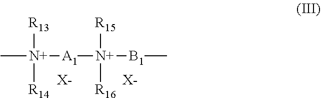

in which:
R₁₃, R₁₄, R₁₅ and R₁₆, which are identical or different, represent aliphatic, alicyclic or arylaliphatic radicals comprising from 1 to 20 carbon atoms or C₁-C₁₂ hydroxyalkyl aliphatic radicals;
or else R₁₃, R₁₄, R₁₅ and R₁₆, together or separately, constitute, with the nitrogen atoms to which they are attached, heterocycles optionally comprising a second heteroatom other than nitrogen;
or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group, where R₁₇ is an alkylene and D is a quaternary ammonium group;
A₁ and B₁ represent saturated or unsaturated, linear or branched, divalent polymethylene groups comprising from 2 to 20 carbon atoms and which can contain, bonded to or intercalated in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a saturated or unsaturated, linear or branched, alkylene or hydroxyalkylene radical, B₁ can also denote a —(CH₂)ₙ—CO-D-OC—(CH₂)ₚ— group with n and p, which are identical or different, being integers varying from 2 to 20, and D denoting:
   a) a glycol residue of formula —O—Z—O—, where Z denotes a linear or branched hydrocarbon radical or a group corresponding to one of the following formulae: —(CH₂CH₂O)_{X}CH₂CH₂— and —[CH₂CH(CH₃)O]_{y}—CH₂CH(CH₃)—, where x and y denote an integer from 1 to 4, representing a defined and unique degree of polymerization or any number from 1 to 4 representing a mean degree of polymerization;
   b) a bis-secondary diamine residue, such as a piperazine derivative;
   c) a bis-primary diamine residue of formula —NH—Y—NH—, where Y denotes a linear or branched hydrocarbon radical, or else the divalent radical —CH₂—CH₂—S—S—CH₂—CH₂—;
   d) a ureylene group of formula —NH—CO—NH—.

Preferably, X⁻ is an anion, such as chloride or bromide. These polymers have a number-average molar mass (Mn) generally of between 1000 and 100 000.

Mention may more particularly be made of polymers which are constituted of repeat units corresponding to the formula:

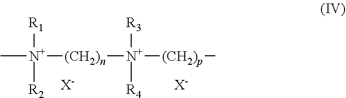

in which R₁, R₂, R₃ and R₄, which are identical or different, denote an alkyl or hydroxyalkyl radical having from 1 to 4 carbon atoms, n and p are integers varying from 2 to 20 and X⁻ is an anion derived from a mineral or organic acid.

A particularly preferred compound of formula (IV) is that for which R₁, R₂, R₃ and R₄ represent a methyl radical, n=3, p=6 and X=Cl, known as Hexadimethrine Chloride according to the INCI (CTFA) nomenclature.
(9) polyquaternary ammonium polymers comprising units of formula (V):

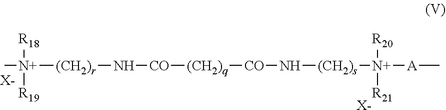

in which:
R₁₈, R₁₉, R₂₀ and R₂₁, which are identical or different, represent a hydrogen atom or a methyl, ethyl, propyl, P-hydroxyethyl, P-hydroxypropyl or —CH₂CH₂(OCH₂CH₂)ₚOH radical, in which p is equal to 0 or to an integer of between 1 and 6, with the proviso that R₁₈, R₁₉, R₂₀ and R₂₁ do not simultaneously represent a hydrogen atom,
r and s, which are identical or different, are integers of between 1 and 6,
q is equal to 0 or to an integer of between 1 and 34,
X⁻ denotes an anion, such as a halide,
A denotes a divalent radical of a dihalide or preferably represents —CH₂—CH₂—O—CH₂—CH₂—.

Mention may be made, for example, of the products Mirapol® A 15, Mirapol® AD1, Mirapol® AZ1 and Mirapol® 175 sold by Miranol;
(10) quaternary polymers of vinylpyrrolidone and of vinylimidazole, such as, for example, the products sold under the names Luviquat® FC 905, FC 550 and FC 370 by BASF;
(11) polyamines, such as Polyquart® H sold by Cognis, which is referenced under the name Polyethylene Glycol (15) Tallow Polyamine in the CTFA dictionary;
(12) polymers comprising, in their structure:
(a) one or more units corresponding to the following formula (A):

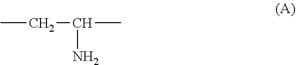

(b) optionally one or more units corresponding to the following formula (B):

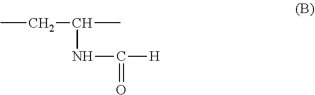

In other words, these polymers can be chosen in particular from homopolymers or copolymers comprising one or more units resulting from vinylamine and optionally one or more units resulting from vinylformamide.

Preferably, these cationic polymers are chosen from polymers comprising, in their structure, from 5 mol % to 100 mol % of units corresponding to the formula (A) and from 0 mol % to 95 mol % of units corresponding to the formula (B), preferentially from 10 mol % to 100 mol % of units corresponding to the formula (A) and from 0 mol % to 90 mol % of units corresponding to the formula (B).

These polymers can be obtained, for example, by partial hydrolysis of polyvinylformamide. This hydrolysis can take place in an acidic or basic medium. The weight-average molecular weight of said polymer, measured by light scattering, can range from 1000 to 3 000 000 g/mol, preferably from 10 000 to 1 000 000 g/mol and more particularly from 100 000 to 500 000 g/mol.

The polymers comprising units of formula (A) and optionally units of formula (B) are in particular sold under the name Lupamin by BASF, such as, for example, and in a non-limiting way, the products sold under the names Lupamin 9095, Lupamin 5095, Lupamin 1095, Lupamin 9030 (or Luviquat 9030) and Lupamin 9010.

Preferably, the cationic polymers capable of being employed in the context of the invention are chosen, alone or as a mixture, from the polymers of the family (1) and/or the cationic polysaccharides, in particular cationic celluloses, such as Polyquaternium-10; cationic galactomannan gums, in particular cationic guar gums; and also their mixtures.

When they are present, the composition according to the invention can comprise the cationic polymer(s) in a total amount ranging from 0.01% to 10% by weight, better still from 0.05% to 5% by weight, even better still from 0.1% to 2% by weight, with respect to the total weight of the composition.

When they are present, the composition according to the invention can comprise the cationic polysaccharide(s) in a total amount ranging from 0.01% to 10% by weight, better still from 0.05% to 5% by weight, even better still from 0.1% to 2% by weight, with respect to the total weight of the composition.

### Non-Ionic Polysaccharides

The composition used according to the invention can optionally comprise one or more non-ionic polysaccharides other than the associative polymers described above. This or these non-ionic polysaccharides are thus non-associative.

The non-ionic polysaccharides are preferably chosen, alone or as a mixture, from celluloses, starches, galactomannans and their non-ionic derivatives, in particular their ethers or esters.

These polymers can be physically or chemically modified. Mention may be made, as physical treatment, of the temperature and mention may be made, as chemical treatment, of the esterification, etherification, amidation and oxidation reactions, in so far as these treatments make it possible to result in polymers which are non-ionic.

Mention may be made, as galactomannans capable of being used, of non-ionic guar gums which can be modified with (poly)hydroxy(C₁-C₆)alkyl groups, in particular hydroxymethyl, hydroxyethyl, hydroxypropyl and hydroxybutyl groups.

These guar gums are well known from the state of the art and can be prepared, for example, by reacting corresponding alkene oxides, such as, for example, propylene oxides, with the guar gum so as to obtain a guar gum modified with hydroxypropyl groups.

The degree of hydroxyalkylation preferably varies from 0.4 to 1.2 and corresponds to the number of alkylene oxide molecules consumed by the number of free hydroxyl functions present on the guar gum.

Such non-ionic guar gums optionally modified with hydroxyalkyl groups are, for example, sold under the trade names Jaguar HP8, Jaguar HP60, Jaguar HP120, Jaguar HP105 SGI and Jaguar HP8 SGI by Rhodia Chimie.

The starch molecules which can be used in the present invention can have, as botanical source, cereals or also tubers. Thus, the starches are chosen, for example, from maize starch, rice starch, cassava starch, barley starch, potato starch, wheat starch, sorghum starch or pea starch. The starches can be chemically or physically modified, in particular by one or more of the following reactions: pregelatinization, oxidation, crosslinking, esterification, etherification, amidation or heat treatments.

The starch molecules can result from any plant source of starch, such as in particular maize, potato, oats, rice, tapioca, sorghum, barley or wheat. It is also possible to use hydrolysates of the abovementioned starches. The starch preferably results from potato.

The non-ionic polysaccharides can also be cellulose-based polymers not comprising a C₁₀-C₃₀ fatty chain in their structure.

“Cellulose-based” polymer is understood to mean, according to the invention, any polysaccharide compound possessing, in its structure, sequences of glucose residues linked together by β-1,4 bonds; the cellulose-based polymers can be unsubstituted celluloses, and/or derivatives of non-ionic celluloses.

Thus, the cellulose-based polymers which can be used according to the invention can be chosen from unsubstituted celluloses, including under a microcrystalline form, and cellulose ethers. Among these cellulose-based polymers, cellulose ethers, cellulose esters and cellulose ether/esters are distinguished.

Mention may be made, among non-ionic cellulose ethers, of (C₁-C₄)alkylcelluloses, such as methylcelluloses and ethylcelluloses (for example Ethocel Standard 100 Premium from Dow Chemical); (poly)hydroxy(C₁-C₄)alkylcelluloses, such as hydroxymethylcelluloses, hydroxyethylcelluloses (for example Natrosol 250 HHR provided by Aqualon) and hydroxypropylcelluloses (for example Klucel EF from Aqualon); mixed (poly)hydroxy(C₁-C₄)alkyl(C₁-C₄)alkylcelluloses, such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Preferably, the non-ionic polysaccharides are chosen, alone or as a mixture, from celluloses, galactomannans and their non-ionic derivatives, in particular their ethers; and better still, alone or as a mixture, from non-ionic guar gums optionally modified with (poly)hydroxy(C₁-C₆)alkyl, in particular hydroxypropyl, groups; and/or celluloses, which are substituted or unsubstituted, and cellulose ethers, such as (C₁-C₄)alkylcelluloses and (poly)hydroxy(C₁-C₄)alkylcelluloses.

Preferably, the non-ionic polysaccharides are chosen, alone or as a mixture, from non-ionic guar gums optionally modified with (poly)hydroxy(C₁-C₆)alkyl, in particular hydroxypropyl (INCI name: Hydroxypropyl Guar), groups.

When they are present, the composition according to the invention can comprise the non-ionic polysaccharide(s) in a total amount ranging from 0.01% to 10% by weight, better still from 0.05% to 5% by weight, even better still from 0.1% to 2% by weight, with respect to the total weight of the composition.

When they are present, the composition according to the invention can comprise the non-ionic polysaccharide(s) chosen from celluloses, galactomannans and their non-ionic derivatives, in particular their ethers, and their mixtures, in a total amount ranging from 0.01% to 10% by weight, better still from 0.05% to 5% by weight, even better still from 0.1% to 2% by weight, with respect to the total weight of the composition.

In a preferred embodiment, the composition according to the invention can comprise one or more cationic polymers and one or more non-ionic polysaccharides; in particular one or more cationic polysaccharides and one or more non-ionic polysaccharides; better still one or more cationic polysaccharides chosen from cationic guar gums and cationic celluloses, and one or more non-ionic guar gums.

### Non-Ionic Surfactants

The composition used according to the invention can optionally comprise one or more non-ionic surfactants.

Mention may be made, as examples of non-ionic surfactants, of the following compounds, alone or as a mixture:
oxyalkylenated (C₈-C₂₄)alkylphenols;
saturated or unsaturated, linear or branched, oxyalkylenated or glycerolated, C₈-C₄₀ alcohols; they preferably comprise one or two fatty chains;
saturated or unsaturated, linear or branched, oxyalkylenated C₈ to Co fatty acid amides;
esters of saturated or unsaturated, linear or branched, C₈ to Co acids and of polyethylene glycols;
esters of saturated or unsaturated, linear or branched, C₈ to Co acids and of sorbitol which are preferably oxyethylenated;
esters of fatty acids and of sucrose;
optionally oxyalkylenated alkyl (poly)glycosides (0 to 10 oxyalkylene units) which can comprise from 1 to 15 glucose units;
saturated or unsaturated oxyethylenated vegetable oils;
condensates of ethylene oxide and/or of propylene oxide;
N—(C₈-C₅₀)alkylglucamine and N—(C₈-C₅₀)acylmethylglucamine derivatives;
amine oxides.

The oxyalkylene units are more particularly oxyethylene or oxypropylene units, or their combination, preferably oxyethylene units.

The number of moles of ethylene oxide and/or of propylene oxide preferably ranges from 1 to 250, more particularly from 2 to 100 and better still from 2 to 50; the number of moles of glycerol ranges in particular from 1 to 50 and better still from 1 to 10.

Advantageously, the non-ionic surfactants according to the invention do not comprise oxypropylene units.

Preferably, they comprise a number of moles of ethylene oxide ranging from 1 to 250, in particular from 2 to 100 and better still from 2 to 50.

Use is preferably made, as examples of glycerolated non-ionic surfactants, of mono- or polyglycerolated C₈ to C₄₀ alcohols comprising from 1 to 50 mol of glycerol and preferably from 1 to 10 mol of glycerol.

Mention may be made of lauryl alcohol having 4 mol of glycerol (INCI name: Polyglyceryl-4 Lauryl Ether), lauryl alcohol having 1.5 mol of glycerol, oleyl alcohol having 4 mol of glycerol (INCI name: Polyglyceryl-4 Oleyl Ether), oleyl alcohol having 2 mol of glycerol (INCI name: Polyglyceryl-2 Oleyl Ether), cetearyl alcohol having 2 mol of glycerol, cetearyl alcohol having 6 mol of glycerol, oleyl/cetyl alcohol having 6 mol of glycerol and octadecanol having 6 mol of glycerol.

Among the glycerolated alcohols, it is more particularly preferred to use the C₈ to C₁₀ alcohol having 1 mol of glycerol, the C₁₀ to C₁₂ alcohol having 1 mol of glycerol and the C₁₂ alcohol having 1.5 mol of glycerol.

Non-ionic surfactants of alkyl (poly)glycoside type can in particular be represented by the following general formula: R₁O—(R₂₀)ₜ-(G)ᵥ, in which:
R₁ represents a linear or branched alkyl or alkenyl radical comprising from 6 to 24 carbon atoms, in particular from 8 to 18 carbon atoms, or an alkylphenyl radical, the linear or branched alkyl radical of which comprises from 6 to 24 carbon atoms, in particular from 8 to 18 carbon atoms;
R₂ represents an alkylene radical comprising from 2 to 4 carbon atoms;
G represents a sugar unit comprising from 5 to 6 carbon atoms;
t denotes a value ranging from 0 to 10, preferably from 0 to 4;
v denotes a value ranging from 1 to 15, preferably from 1 to 4.

Preferably, the alkyl (poly)glycoside surfactants are compounds of the formula described above in which:
R₁ denotes a saturated or unsaturated, linear or branched, alkyl radical comprising from 8 to 18 carbon atoms,
R₂ represents an alkylene radical comprising from 2 to 4 carbon atoms,
t denotes a value ranging from 0 to 3, preferably equal to 0,
G denotes glucose, fructose or galactose, preferably glucose,
it being possible for the degree of polymerization, that is to say the value of v, to range from 1 to 15, preferably from 1 to 4; the mean degree of polymerization more particularly being of between 1 and 2.

The glucoside bonds between the sugar units are generally of 1-6 or 1-4 type, preferably of 1-4 type. Preferably, the alkyl (poly)glycoside surfactant is an alkyl (poly)glucoside surfactant. Preference is very particularly given to C₈/C₁₆ alkyl (poly)glucosides of 1-4 type and in particular decyl glucosides and caprylyl/capryl glucosides.

Mention may be made, among the commercial products, of the products sold by Cognis under the names Plantaren® (600 CS/U, 1200 and 2000) or Plantacare® (818, 1200 and 2000); the products sold by SEPPIC under the names Oramix CG 110 and Oramix® NS 10; the products sold by BASF under the name Lutensol GD 70 or also the products sold by Chem Y under the name AG10 LK.

Preferably, use is made of C₈/C₁₆ alkyl (poly)glycosides of 1-4 type, in particular as a 53% aqueous solution, such as those sold by Cognis under the reference Plantacare® 818 UP.

The non-ionic surfactant(s) used in the composition according to the invention are preferentially chosen, alone or as a mixture, from:
saturated or unsaturated, linear or branched, oxyethylenated C₈ to C₄₀ alcohols comprising from 1 to 100 mol of ethylene oxide, preferably from 2 to 50 mol of ethylene oxide and more particularly from 2 to 40 mol of ethylene oxide; they preferably comprise one or two fatty chains;
saturated or unsaturated oxyethylenated vegetable oils comprising from 1 to 100 mol and preferably from 2 to 50 mol of ethylene oxide;
(C₈-C₃₀)alkyl (poly)glycosides which are optionally oxyalkylenated, preferably with from 0 to 10 mol of ethylene oxide, and which comprise from 1 to 15 glucose units;
mono- or polyglycerolated C₈ to C₄₀ alcohols comprising from 1 to 50 mol of glycerol, preferably from 1 to 10 mol of glycerol;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
esters of saturated or unsaturated, linear or branched, C₈ to C₃₀ acids and of sorbitol which are preferably oxyethylenated;

More preferentially, the non-ionic surfactant(s) used in the composition according to the invention are chosen, alone or as a mixture, from:
saturated or unsaturated, linear or branched, oxyethylenated C₈ to C₄₀ alcohols comprising from 1 to 100 mol of ethylene oxide, preferably from 2 to 50 mol of ethylene oxide and more particularly from 2 to 40 mol of ethylene oxide and comprising one or two fatty chains, in particular at least one C₈-C₂₀, in particular C₁₀-C₁₈, alkyl chain;
esters of saturated or unsaturated, linear or branched, C₈ to C₃₀ acids and of sorbitol which are preferably oxyethylenated; and
(C₈-C₃₀)alkyl (poly)glucosides which are optionally oxyalkylenated, preferably comprising from 0 to 10 mol of ethylene oxide, and which comprise from 1 to 15 glucose units.

Preferably, the composition according to the invention comprises the non-ionic surfactant(s) in a total content ranging from 0.05% to 10% by weight, preferably from 0.1% to 5% by weight, preferentially from 0.2% to 3% by weight, with respect to the total weight of the composition according to the invention.

### Fatty Substances

The composition used according to the invention can optionally comprise one or more non-silicone fatty substances, which can be chosen from solid fatty substances, liquid fatty substances and their mixtures.

“Non-silicone fatty substance” is understood to mean a fatty substance not containing Si—O bonds.

“Solid fatty substance” is understood to mean a fatty substance having a melting point of greater than 25° C., preferably of greater than or equal to 28° C., preferably of greater than or equal to 30° C., at atmospheric pressure (1.013×10⁵ Pa). Advantageously, the solid fatty substances which can be used in the present invention are neither (poly)oxyalkylenated nor (poly)glycerolated.

The solid fatty substances can be chosen from solid fatty acids, solid fatty alcohols, solid esters of fatty acids and/or of fatty alcohols, waxes and ceramides, and their mixtures.

“Fatty acid” is understood to mean a carboxylic acid having a long chain comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated. The solid fatty acids which can be used in the present invention are in particular chosen from myristic acid, cetylic acid, stearylic acid, palmitic acid, stearic acid, lauric acid, behenic acid and their mixtures. Said fatty acids are different from the (poly)hydroxylated carboxylic acids comprising from 2 to 8 carbon atoms described above.

“Fatty alcohol” is understood to mean an aliphatic alcohol having a long chain comprising from 6 to 40 carbon atoms, preferably from 8 to 30 carbon atoms, and comprising at least one hydroxyl OH group. These fatty alcohols are neither oxyalkylenated nor glycerolated. The solid fatty alcohols can be saturated or unsaturated and linear or branched and they comprise from 8 to 40 carbon atoms, preferably from 10 to 30 carbon atoms, better still from 12 to 30 carbon atoms. Preferably, the solid fatty alcohols are of structure R—OH with R denoting a linear alkyl group, optionally substituted with one or more hydroxyl groups, comprising from 8 to 40 carbon atoms, preferentially from 10 to 30 carbon atoms, better still from 12 to 30 carbon atoms, indeed even from 12 to 24 carbon atoms and even better still from 14 to 22 carbon atoms. The solid fatty alcohols capable of being used are preferably chosen from saturated, linear or branched, preferably saturated and linear, (mono)alcohols comprising from 8 to 40 carbon atoms, better still from 10 to 30 carbon atoms, indeed even from 12 to 24 carbon atoms and better still from 14 to 22 carbon atoms.

The solid fatty alcohols capable of being used can be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol and their mixtures, such as cetylstearyl or cetearyl alcohol. Particularly preferably, the solid fatty alcohol is chosen from cetyl alcohol, stearyl alcohol or their mixtures, such as cetylstearyl alcohol; better still, the solid fatty alcohol is cetylstearyl alcohol.

The solid esters of a fatty acid and/or of a fatty alcohol capable of being used are preferably chosen from the esters resulting from C₉-C₂₆ fatty carboxylic acid and/or from C₉-C₂₆ fatty alcohol.

Preferably, these solid fatty esters are esters of a saturated, linear or branched, carboxylic acid comprising at least 10 carbon atoms, preferably from 10 to 30 carbon atoms and more particularly from 12 to 24 carbon atoms and of a saturated, linear or branched, monoalcohol comprising at least 10 carbon atoms, preferably from 10 to 30 carbon atoms and more particularly from 12 to 24 carbon atoms. The saturated carboxylic acids can optionally be hydroxylated and are preferably monocarboxylic acids.

Use may also be made of esters of C₄-C₂₂ di- or tricarboxylic acids and of C₁-C₂₂ alcohols and of esters of mono-, di- or tricarboxylic acids and of di-, tri-, tetra- or pentahydroxylated C₂-C₂₆ alcohols.

Mention may in particular be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate and their mixtures.

Preferably, the solid esters of a fatty acid and/or of a fatty alcohol are chosen from C₉-C₂₆ alkyl palmitates, in particular myristyl, cetyl or stearyl palmitate; C₉-C₂₆ alkyl myristates, such as cetyl myristate, stearyl myristate and myristyl myristate; and C₉-C₂₆ alkyl stearates, in particular myristyl, cetyl and stearyl stearate; and their mixtures.

Particularly preferably, the solid esters of a fatty acid and/or of a fatty alcohol are chosen from myristyl stearate, myristyl palmitate and their mixtures.

A wax, within the meaning of the present invention, is a lipophilic compound, which is solid at 25° C. and atmospheric pressure, with a reversible solid/liquid change in state, having a melting point of greater than approximately 40° C. and which can range up to 200° C., and exhibiting, in the solid state, an anisotropic crystal organization. In general, the size of the crystals of wax is such that the crystals diffract and/or scatter light, conferring on the composition which comprises them a cloudy, more or less opaque, appearance. On bringing the wax to its melting point, it is possible to render it miscible with oils and to form a microscopically homogeneous mixture but, on returning the temperature of the mixture to ambient temperature, recrystallization of the wax, which is detectable microscopically and macroscopically (opalescence), is obtained.

In particular, the waxes which are suitable for the invention can be chosen from waxes of animal, vegetable or mineral origin, synthetic non-silicone waxes and their mixtures.

Mention may in particular be made of hydrocarbon waxes, such as beeswax or modified beeswaxes (cera bellina), lanolin wax and lanolin derivatives, spermaceti; cork fibre or sugar cane waxes, olive tree wax, rice bran wax, carnauba wax, candelilla wax, ouricury wax, esparto grass wax, berry wax, shellac wax, Japan wax and sumac wax; absolute waxes of flowers; montan wax, orange wax and lemon wax, microcrystalline waxes, paraffins, petrolatum, lignite and ozokerite; polyethylene waxes, the waxes obtained by the Fischer-Tropsch synthesis and waxy copolymers, and also their esters.

Mention may additionally be made of C₂₀ to C₆₀ microcrystalline waxes, such as Microwax HW.

Mention may also be made of the MW 500 polyethylene wax sold under the reference Permalen 50-L Polyethylene.

Mention may also be made of waxes obtained by catalytic hydrogenation of animal or vegetable oils having linear or branched C₈ to C₃₂ fatty chains. Mention may in particular be made, among the latter, of isomerized jojoba oil, such as trans-isomerized partially hydrogenated jojoba oil, in particular that manufactured or sold by Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane) tetrastearate, in particularthat sold under the name Hest 2T-4S® by Heterene.

Use may also be made of the waxes obtained by hydrogenation of castor oil esterified with cetyl alcohol, such as those sold under the names Phytowax Castor 16L64® and 22L73® by Sophim.

Use may also be made, as wax, of a C₂₀ to C₄₀ alkyl (hydroxystearyloxy)stearate (the alkyl group comprising from 20 to 40 carbon atoms), alone or as a mixture.

Such a wax is in particular sold under the names Kester Wax K 82 P®, Hydroxypolyester K 82 P® and Kester Wax K 80 P® by Koster Keunen.

It is also possible to use microwaxes in the compositions of the invention; mention may in particular be made of carnauba microwaxes, such as that sold under the name MicroCare 350® by Micro Powders, synthetic wax microwaxes, such as that sold under the name MicroEase 114S® by Micro Powders, microwaxes constituted of a mixture of carnauba wax and of polyethylene wax, such as those sold under the names Micro Care 300® and 310® by Micro Powders, microwaxes constituted of a mixture of carnauba wax and of synthetic wax, such as that sold under the name Micro Care 325® by Micro Powders, polyethylene microwaxes, such as those sold under the names Micropoly 200®, 220®, 220L® and 250S® by Micro Powders, and polytetrafluoroethylene microwaxes, such as those sold under the names Microslip 519® and 519 L® by Micro Powders.

The waxes are preferably chosen from mineral waxes, such as paraffin, petrolatum, lignite or ozokerite wax; vegetable waxes, such as cocoa butter, shea butter, cork fibre or sugar cane waxes, olive tree wax, rice bran wax, hydrogenated jojoba wax, ouricury wax, carnauba wax, candelilla wax, esparto grass wax or absolute waxes of flowers, such as the essential wax of blackcurrant blossom sold by Bertin (France); waxes of animal origin, such as beeswaxes or modified beeswaxes (cera bellina), spermaceti, lanolin wax and lanolin derivatives; microcrystalline waxes; and their mixtures.

Ceramides, or ceramide analogues, such as glycoceramides, capable of being used in the compositions according to the invention, are known; mention may in particular be made of ceramides of classes I, II, III and V according to the Dawning classification.

The ceramides or their analogues capable of being employed preferably correspond to the following formula:

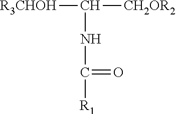

in which:
R₁ denotes a saturated or unsaturated, linear or branched, alkyl group derived from C₁₄-C₃₀ fatty acids, it being possible for this group to be substituted with a hydroxyl group in the a position, or a hydroxyl group in the ω position esterified with a saturated or unsaturated C₁₆-C₃₀ fatty acid;
R₂ denotes a hydrogen atom, a (glycosyl)ₙ group, a (galactosyl)ₘ group or a sulfogalactosyl group, in which n is an integer varying from 1 to 4 and m is an integer varying from 1 to 8;
R₃ denotes a C₁₅-C₂₆ hydrocarbon group which is saturated or unsaturated in the a position, it being possible for this group to be substituted with one or more C₁-C₁₄ alkyl groups;
it being understood that, in the case of natural ceramides or glycoceramides, R₃ can also denote an a-hydroxy(C₁₅-C₂₆)alkyl group, the hydroxyl group being optionally esterified with an a-hydroxy C₁₆-C₃₀ acid.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

The ceramides which are more particularly preferred are the compounds for which R₁ denotes a saturated or unsaturated alkyl derived from C₁₆-C₂₂ fatty acids; R₂ denotes a hydrogen atom and R₃ denotes a saturated or unsaturated linear C₁₅ group.

Use may also be made of the compounds for which R₁ denotes a saturated or unsaturated alkyl radical derived from C₁₂-C₂₂ fatty acids; R₂ denotes a galactosyl or sulfogalactosyl radical and R₃ denotes a saturated or unsaturated C₁₂-C₂₂ hydrocarbon radical and preferably a —CH═CH—(CH₂)₁₂—CH₃ group.

Mention may also be made, as compounds which are particularly preferred, of 2-N-linoleoylaminooctadecane-1,3-diol; 2-N-oleoylaminooctadecane-1,3-diol; 2-N-palmitoylaminooctadecane-1 3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and their mixtures. Preferably, N-oleoyldihydrosphingosine will be used.

Mention may be made, as liquid fatty substances capable of being used, of liquid hydrocarbons, liquid fatty alcohols, liquid esters of fatty acids and/or fatty alcohols other than the triglycerides, oils of triglyceride type of vegetable or synthetic origin, mineral oils and their mixtures.

The liquid fatty substances have a melting point of less than or equal to 25° C., preferably of less than or equal to 20° C., at atmospheric pressure (1.013×10⁵ Pa). Advantageously, the liquid fatty substances are not (poly)oxyalkylenated.

It should be remembered that the fatty alcohols, esters and acids more particularly exhibit at least one saturated or unsaturated, linear or branched, hydrocarbon group comprising from 6 to 40 and better still from 8 to 30 carbon atoms which is optionally substituted, in particular with one or more (in particular from 1 to 4) hydroxyl groups. If they are unsaturated, these compounds can comprise from one to three conjugated or non-conjugated carbon-carbon double bonds.

The liquid hydrocarbons can be C₆ to C₁₈ liquid hydrocarbons and be linear, branched or optionally cyclic; they are preferably chosen from C₈-C₁₆, in particular C₁₀-C₁₄, alkanes. Mention may be made, by way of example, of hexane, cyclohexane, undecane, dodecane, isododecane, tridecane, isoparaffins, such as isohexadecane or isodecane, and their mixtures.

The liquid hydrocarbons can also be chosen from those comprising more than 16 carbon atoms, which can be linear or branched, of mineral or synthetic origin; mention may be made of liquid paraffins or liquid petrolatum, polydecenes, hydrogenated polyisobutene, such as Parleam®, and their mixtures.

The triglyceride oils of vegetable or synthetic origin can be chosen from liquid triglycerides of fatty acids comprising from 6 to 30 carbon atoms, such as triglycerides of heptanoic acid or octanoic acid, or also, for example, sunflower oil, maize oil, soybean oil, marrow oil, grapeseed oil, sesame seed oil, hazelnut oil, apricot oil, macadamia oil, arara oil, castor oil, avocado oil, triglycerides of caprylic/capric acids, such as those sold by Stearinerie Dubois or those sold under the names Miglyol® 810, 812 and 818 by Dynamit Nobel, jojoba oil, shea butter oil and their mixtures.

The liquid fatty alcohols can be chosen from saturated or unsaturated and linear or branched, preferably unsaturated or branched, alcohols comprising from 6 to 40 carbon atoms, preferably from 8 to 30 carbon atoms. Mention may be made, for example, of octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and their mixtures.

The liquid esters of fatty acids and/or fatty alcohols other than the triglycerides mentioned above, can be chosen from esters of saturated or unsaturated, linear C₁ to C₂₆ or branched C₃ to C₂₆, aliphatic mono- or polyacids and of saturated or unsaturated, linear C₁ to C₂₆ or branched C₃ to C₂₆, aliphatic mono- or polyalcohols, the total carbon number of the esters being greater than or equal to 6, more advantageously greater than or equal to 10.

Preferably, for the esters of monoalcohols, one at least of the alcohol or of the acid from which the esters of the invention result is branched.

Mention may be made, among the monoesters, of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; alkyl palmitates such as ethyl or isopropyl palmitates, or 2-ethylhexyl palmitate or 2-octyldecyl palmitate; alkyl myristates, such as isopropyl or 2-octyldodecyl myristate; isobutyl stearate; 2-hexyldecyl laurate and their mixtures. Preferably, among the monoesters of monoacids and of monoalcohols, use will be made of ethyl palmitate or isopropyl palmitate, alkyl myristates, such as isopropyl or ethyl myristate, isocetyl stearate, 2-ethylhexyl isononanoate, isodecyl neopentanoate, isostearyl neopentanoate and their mixtures.

Still within the context of this alternative form, use may also be made of esters of C₄ to C₂₂ di- or tricarboxylic acids and of C₁ to C₂₂ alcohols and of esters of mono-, di- or tricarboxylic acids and of C₂ to C₂₆ di-, tri-, tetra- or pentahydroxylated alcohols. Mention may in particular be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; polyethylene glycol distearates and their mixtures.

The composition can also comprise, as fatty ester, sugar esters and diesters of C₆ to C₃₀, preferably C₁₂ to C₂₂, fatty acids. It should be remembered that “sugar” is understood to mean oxygen-comprising hydrocarbon compounds which possess several alcohol functions, with or without aldehyde or ketone function, and which comprise at least 4 carbon atoms. These sugars can be monosaccharides, oligosaccharides or polysaccharides.

Mention may be made, as suitable sugars, for example, of sucrose, glucose, galactose, ribose, fucose, maltose, fructose, mannose, arabinose, xylose, lactose and their derivatives, in particular alkyl derivatives, such as methyl derivatives, for instance methylglucose.

The sugar esters of fatty acids can be chosen in particular from the group comprising the esters or mixtures of esters of sugars described above and of saturated or unsaturated, linear or branched, C₆ to C₃₀, preferably C₁₂ to C₂₂, fatty acids. If they are unsaturated, these compounds can comprise from one to three conjugated or non-conjugated carbon-carbon double bonds.

The esters according to this alternative form can also be chosen from mono-, di-, tri- and tetraesters, polyesters and their mixtures.

These esters can, for example, be oleates, laurates, palmitates, myristates, behenates, cocoates, stearates, Iinoleates, Iinolenates, caprates, arachidonates or their mixtures, such as in particular the oleate/palmitate, oleate/stearate and palmitate/stearate mixed esters.

More particularly, use is made of mono- and diesters and in particular sucrose, glucose or methylglucose mono- or dioleates, -stearates, -behenates, -oleate/palmitates, -linoleates, -linolenates or -oleate/stearates, and their mixtures.

Mention may be made, by way of example, of the product sold under the name Glucate® DO by Amerchol, which is a methylglucose dioleate.

Preferably, use will be made of a liquid ester of a monoacid and of a monoalcohol.

Preferably, the fatty substances are chosen from triglyceride oils of vegetable or synthetic origin, liquid esters of a fatty acid and/or a fatty alcohol other than the triglycerides, liquid C₆-C₁₈ hydrocarbons, solid fatty alcohols, solid esters of fatty acids and/or of fatty alcohols, and their mixtures.

Preferably, the composition according to the invention can comprise the fatty substance(s) in a total amount ranging from 0.1% to 20% by weight, better still from 1% to 18% by weight, preferentially from 2% to 15% by weight, even better still from 5% to 12% by weight, with respect to the total weight of the composition.

### Additional Compounds

The composition used according to the invention advantageously comprises water, in particular at a concentration preferably ranging from 50% to 95% by weight, for example from 55% to 90% by weight, in particular from 60% to 85% by weight, better still from 65% to 85% by weight, with respect to the total weight of the composition. The pH of the composition can be between 2.5 and 8, preferentially between 3 and 7, indeed even between 4 and 6.

The composition used according to the invention can optionally comprise one or more preferably hydrophilic (water-soluble or water-miscible) organic solvents which are liquid at 25° C., 1 atm, which can be chosen from C₁-C₆ aliphatic or aromatic monoalcohols, C₂-C₈ polyols and C₃-C₇ polyol ethers. Advantageously, the organic solvent is chosen from C₂-C₄ mono-, di- or tridiols. It can advantageously be chosen from ethanol, isopropanol, benzyl alcohol, glycerol, 1,2-propanediol (propylene glycol) and their mixtures.

The composition used according to the invention can additionally comprise at least one or more standard cosmetic ingredients chosen in particular from thickeners, gelling agents, both different from the polymers described above; sunscreens; anti-dandruff agents; antioxidants; chelating agents; reducing agents; oxidation bases, couplers, oxidizing agents, direct dyes; hair-straightening agents; pearlescent and opacifying agents; micas, pearlescent agents, glitter; plasticizing or coalescence agents; pigments; fillers; fragrances; basifying or acidifying agents; silanes. A person skilled in the art will take care to choose the ingredients participating in the composition, and also their amounts, so that they do not harm the properties of the compositions of the present invention.

The composition used according to the invention can in addition advantageously comprise one or more silicone polymers derived from an amino acid, in particular of following formula (A1) or (A2):

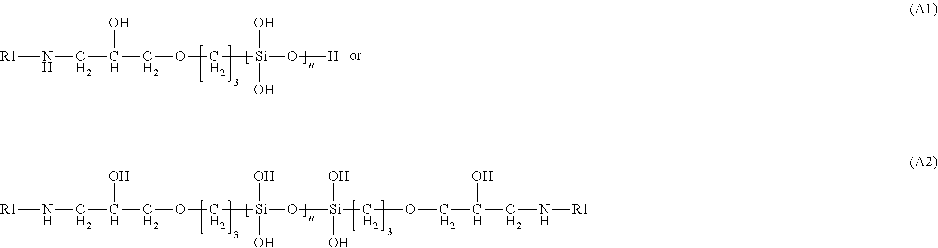

in which:
n is an integer of between 1 and 100, preferably between 1 and 50, more preferentially between 1 and 20, better still between 1 and 10, even better still between 1 and 5, even better still between 1 and 3,
R₁ is a polypeptide chain derived from an amino acid, such as cysteine.

Preferably, the silicone polymer(s) derived from an amino acid correspond to the formula (A1) in which R₁ is a hydrolysed plant protein residue.

Mention may in particular be made of the compounds having as INCI name: Hydrolyzed Wheat Protein PG-Propyl Silanetriol, Hydrolyzed Vegetable Protein PG-Propyl Silanetriol or Cystine Bis-PG-Propyl Silanetriol, which are sold under the names Crodasone® W, Crodasone® W PF, Keravis® PE or Crodasone® Cystine by Croda.

Preferably, the silicone polymer(s) derived from an amino acid can be present in the composition according to the invention at a content which can range from 0.1% to 2% by weight, in particular from 0.2% to 1% by weight, with respect to the total weight of the composition.

Better still, the silicone polymer(s) derived from an amino acid of formula (A1) in which R₁ is a hydrolysed plant protein residue can be present in the composition according to the invention at a content which can range from 0.1% to 2% by weight, in particular from 0.2% to 1% by weight, with respect to the total weight of the composition.

According to a preferred embodiment of the invention, the cosmetic composition used in the context of the invention can comprise:
one or more compounds of amino acids type corresponding to the formula (I) as defined above, in which p=2 and R represents a hydrogen atom or a saturated, linear or branched, (C₁-C₄)alkyl group, optionally interrupted by a —S— heteroatom and/or optionally substituted with one or two groups chosen from hydroxyl, amino or —NH—C(NH)—NH₂; better still, R represents a hydrogen atom;
   preferably present in a total content from 0.5% to 10% by weight, in particular from 0.7% to 8% by weight, better still from 0.8% to 7% by weight, with respect to the total weight of the composition;
one or more hydroxylated (poly)carboxylic acids, comprising from 4 to 6 carbon atoms, from 1 to 3 OH groups and 2 or 3 COOH groups, and/or their salts, preferably present in a total content from 0.5% to 10% by weight, with respect to the total weight of the composition, in particular from 1% to 8% by weight, better still from 1.5% to 6% by weight;
optionally one or more associative polymers which are preferably non-ionic, better still chosen from polyether polyurethanes;
   preferably present in the composition in a total content ranging from 0.01% to 10% by weight, preferentially from 0.05% to 5% by weight, more preferentially from 0.1% to 1.5% by weight, with respect to the total weight of the composition;
optionally one or more silicones, preferably chosen from aminated silicones;
preferably present in a total content ranging from 0.3% to 5% by weight, better still from 0.5% to 4% by weight, preferentially from 0.6% to 3% by weight and more preferentially from 0.7% to 2.5% by weight, with respect to the total weight of the composition;
optionally one or more cationic surfactants preferably chosen from those of formula (II) above, those of formula (V) above, those of formula (VI) above and their mixtures, better still from those of formulae (II) and/or (VI) above; preferably present in a total amount ranging from 0.1% to 10% by weight, better still from 0.2% to 8% by weight, preferentially from 0.3% to 7% by weight, even better still from 0.5% to 5% by weight, with respect to the total weight of the composition;
optionally one or more cationic polysaccharides, in particular chosen from celluloses and/or cationic galactomannan gums; preferably present in a total amount ranging from 0.01% to 10% by weight, better still from 0.05% to 5% by weight, even better still from 0.1% to 2% by weight, with respect to the total weight of the composition;
optionally one or more non-ionic polysaccharides, in particular chosen, alone or as a mixture, from non-ionic guar gums optionally modified with (poly)hydroxy(C₁-C₆)alkyl groups; preferably present in a total amount ranging from 0.01% to 10% by weight, better still from 0.05% to 5% by weight, even better still from 0.1% to 2% by weight, with respect to the total weight of the composition;
optionally one or more non-ionic surfactants; preferably present in a total content ranging from 0.05% to 10% by weight, preferably from 0.1% to 5% by weight, preferentially from 0.2% to 3% by weight, with respect to the total weight of the composition according to the invention.

The cosmetic composition used according to the invention may in particular be in the form of a lotion, mask or conditioner that is or is not to be rinsed off, and also in the form of a shampoo, in particular a conditioning shampoo, that may be rinsed off, or of a pre-shampoo that is or is not to be rinsed off before applying a shampoo.

The stage of applying the cosmetic composition described above to the hair may be followed by a rinsing stage, for example with water or with a shampoo after an optional leave-on time.

The following examples serve to illustrate the invention without, however, exhibiting a limiting nature.

In the examples which follow, all the amounts are shown, unless otherwise indicated, as percentage by weight of active material (g % AM) with respect to the total weight of the composition.

### EXAMPLE 1

Composition A according to the invention and comparative composition A′ below were prepared from the ingredients shown in the tables below (g % AM):

TABLE 1

| Composition | A | A′ |
|---|---|---|
| Citric Acid | 2.0 | |
| Glycine | 1 | |
| Behentrimonium Chloride | 3 | 3 |
| Hydroxypropyl Guar | 0.15 | 0.15 |
| Aminopropyl Dimethicone | 0.4 | 0.4 |
| Bis(C13-15 Alkoxy) | 0.3 | 0.3 |
| PG-Amodimethicone | | |
| PEG-150/Decyl Alcohol/SMDI | 0.15 | 0.15 |
| Copolymer | | |
| Butyrospermum Parkii (Shea) Butter | 0.5 | 0.5 |
| Cetearyl Alcohol | 7 | 7 |
| Cetyl Esters | 1.5 | 1.5 |
| Isododecane | 0.4 | 0.4 |
| Isopropyl Alcohol | 0.7 | 0.7 |
| Glycerol | 1 | 1 |
| Polyquaternium-37 | 0.1 | 0.1 |
| Propylene Glycol | 0.2 | 0.2 |
| Steareth-100 | 0.25 | 0.25 |
| Preservatives | q.s. | q.s. |
| Water | q.s. for 100% | q.s. for 100% |

The composition A is provided in the form of a cream and can advantageously be used in rinse-out mode after or before a shampoo. This composition can be used for disentangling the hair, providing care, sheen and strength (slightly less suppleness than classic care treatments, body, and a bulk effect).

The hair is soft, hydrated and with greater sheen; the hairs are considered stronger and less inclined to break.

The composition thus exhibits strengthening properties and makes it possible to reduce the amount of calcium contained in the hair fibre application after application.

The strengthening is measured by using the DSC technique.

### (i) Preparation of the Locks

The measurements are carried out on locks bleached beforehand manually and then treated five times (or ten times) according to the following protocol: the lock is washed with a neutral shampoo, is then rinsed, 2 g of test composition are applied per lock of hair of 5.7 g, the care composition is left to stand for 5 minutes and then rinsing is again carried out.

### (ii) Measurement Method

The differential scanning calorimetry (DSC) technique is known to a person skilled in the art as a method which makes it possible to quantify the strengthening of proteins in the cortex of keratin fibres (Kinetics of the changes imparted to the main structural components of human hair by thermal treatment, F.-J. Wortmann and H. Deutz, J. Appl. Polym. Sci., 48, 137 (1993)). The principle of the test is to measure the denaturation temperature of the proteins. It is widely recognized that the greater the denaturation temperature of the proteins, the better is the integrity of the proteins of the cortex, which reflects the reduction in the breakage of the fibres.

The denaturation temperature is directly related to the density of bonding of the proteins of keratins present in the cortex. Thus, the lower the denaturation temperature, the lower the density of bonding of the proteins to one another, the disulfide bridges break and the cortex is damaged. A difference of 2° C. is recognized by a person skilled in the art as a significant modification.

The appliance used to carry out the measurements is a TA Instruments DSC Q20 reference appliance. This appliance measures the energy flow during the heating of the sample. The temperature at which the energy flow is greatest represents the denaturation temperature.

### (iii) Results

The results of the measurements of the denaturation temperature (Td) for each of the locks treated according to the protocol described above are summarized in the following table and correspond to the mean of 3 measurements carried out per lock.

TABLE 2

| | | Standard |
|---|---|---|
| | Td (° C.) | deviation |
| Natural lock (before bleaching treatment) | 153.31 | 1.36 |
| Untreated bleached lock | 138.73 | 0.75 |
| Bleached lock treated 5 times with the | 155.80 | 0.17 |
| composition A | | |
| Bleached lock treated 5 times with the | 142.12 | 0.41 |
| composition A′ | | |
| Bleached lock treated 10 times with the | 162.55 | 0.34 |
| composition A | | |
| Bleached lock treated 10 times with the | 146.42 | 0.19 |
| composition A′ | | |

These results show that the use of the composition according to the invention increases the bonding density of the proteins of keratins present in the cortex of the treated hairs, thus making it possible to repair the damaged hairs.

Furthermore, the denaturation temperature for the locks treated according to the present invention is better than that measured for the natural and undamaged hair, thus showing that the hairs are repaired.

Analysis of the calcium by X-ray fluorescence according to the RC-ANA-MET-1414 method.

These tests are carried out on locks bleached beforehand and then treated ten times according to the following protocol: the lock is washed with a neutral shampoo, is then rinsed, 2 g of test composition are applied per lock of hair of 5.7 g, the care composition is left to stand for 5 minutes and then rinsing is again carried out.

The following results (mean over 2 measurements) are obtained:

TABLE 3

| | Calcium (concentration in ppm) |
|---|---|
| Untreated bleached lock | 16005 +/− 192 |
| Bleached lock treated 10 times with the | 6683 +/− 136 |
| composition A | |
| Bleached lock treated 10 times with the | 13682 +/− 248 |
| composition A′ | |

It is thus found that the concentration of calcium decreases significantly for the locks treated with the invention.

### EXAMPLE 2

The following composition according to the invention was prepared from the ingredients shown in the tables below (g % AM):

TABLE 4

| Composition | B |
|---|---|
| Citric Acid | 3.0 |
| Glycine | 5 |
| Cetrimonium Chloride | 0.5 |
| Quaternium-80 | 0.4 |
| Hydroxypropyltrimonium Hydrolyzed Wheat Protein | 1 |
| Glycerol | 15 |
| PEG-40 Hydrogenated Castor Oil | 0.8 |
| Polysorbate 20 | 0.8 |
| Polysorbate 80 | 0.8 |
| Propylene Glycol | 5 |
| Preservatives | q.s. |
| Water | q.s. for 100% |

The composition exists in the form of a lotion and can advantageously be used without being rinsed out before shampooing or after shampooing and before the use of a mask or a conditioner without being rinsed out.

This composition can be used to the disentangling of the hair, with contribution of care, sheen and strength (body and bulk effect).

The hair is soft, hydrated and with greater sheen; the hairs are considered stronger and less inclined to break.

The composition thus exhibits strengthening properties and makes it possible to reduce the amount of calcium contained in the hair fibre application after application.

The strengthening is measured by DSC, according to the method described above. The measurements are carried out on locks bleached beforehand manually and then treated five (or ten) times according to the following protocol: the lock is washed with a conventional neutral shampoo, is then rinsed, 2 g of composition according to the invention are applied per lock of hair of 5.7 g, then, without rinsing, 2 g of a conventional hair mask are applied, the mask is left to stand for 5 minutes and then rinsing is again carried out.

This routine according to the invention is compared with the following comparative routine (without application of the composition of the invention): the lock is washed with a conventional neutral shampoo, is then rinsed, 2 g of a conventional hair mask (the same as in the routine according to the invention) are applied, the mask is left to stand for 5 minutes and then rinsing is again carried out.

The following results (mean of 3 measurements per lock) are obtained:

TABLE 5

| | | Standard |
|---|---|---|
| | Td (° C.) | deviation |
| Natural lock (before bleaching treatment) | 153.31 | 1.36 |
| Untreated bleached lock | 138.73 | 0.75 |
| Bleached lock treated 5 times with the | 159.7 | 1.40 |
| routine according to the invention | | |
| Bleached lock treated 5 times with the | 140.8 | 0.32 |
| comparative routine | | |

These results show that the use of the composition according to the invention increases the bonding density of the proteins of keratins present in the cortex of the treated hairs, thus making it possible to repair the damaged hairs.

Analysis of the calcium by X-ray fluorescence according to the RC-ANA-MET-1414 method.

These measurements are carried out on the locks treated according to the routine of the invention and according to the comparative routine described above.

The following results are obtained:

TABLE 6

| | Calcium (concentration in ppm) |
|---|---|
| Untreated bleached lock | 10700 +/− 1000 |
| Bleached lock treated 5 times with the | 3974 +/− 247 |
| routine according to the invention | |
| Bleached lock treated 5 times with the | 8542 +/− 528 |
| comparative routine | |
| Bleached lock treated 10 times with the | 3127 +/− 176 |
| routine according to the invention | |
| Bleached lock treated 10 times with the | 7896 +/− 25 |
| comparative routine | |

It is thus found that the concentration of calcium decreases significantly for the locks treated according to the invention.

1-17. (canceled)

18. A method for limiting calcium amount of the hair, comprising applying to the hair a composition comprising:
(i) one or more amino acid compounds of formula (I), salts thereof, or mixtures thereof: 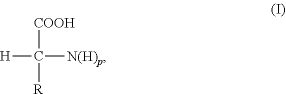
wherein p is an integer equal to 1 or 2,
wherein when p=1, R forms, with the nitrogen atom, a saturated heterocycle comprising from 5 to 8 ring members, substituted by one or more groups chosen from hydroxyl or (C₁-C₄)alkyl, and
wherein when p=2, R represents a hydrogen atom or a saturated, linear or branched, (C₁-C₁₂)alkyl group, optionally interrupted by one or more heteroatoms or groups chosen from —S—, —NH— or —C(NH)— optionally substituted by one or more groups chosen from hydroxyl (—OH), amino (—NH₂), —SH, —COOH, —CONH₂ or —NH—C(NH)—NH₂; and
(ii) one or more hydroxylated (poly)carboxylic acids comprising from 2 to 8 carbon atoms, salts thereof, or mixtures thereof, wherein the total amount of one or more hydroxylated (poly)carboxylic acids comprising from 2 to 8 carbon atoms, salts thereof, or mixtures thereof is at least 0.5% by weight, relative to the total weight of the composition.

19. The method of claim 18, wherein the (i) one or more amino acid compounds of formula (I), salts thereof, or mixtures thereof is chosen from glycine, proline, methionine, serine, arginine, lysine, salts thereof, or mixtures of two or more thereof.

20. The method of claim 18, wherein the total amount of (i) amino acid compounds, salts thereof, or mixtures thereof ranges from 0.5% to 10% by weight, relative to the total weight of the composition.

21. The method of claim 18, wherein the (ii) one or more hydroxylated (poly)carboxylic acids, salts thereof, or mixtures thereof is chosen from lactic acid, glycolic acid, tartaric acid, citric acid, salts thereof, or mixtures of two or more thereof.

22. The method of claim 18, wherein the (ii) one or more hydroxylated (poly)carboxylic acids, salts thereof, or mixtures thereof is chosen from tartaric acid, citric acid, alkali metal salts thereof, alkaline earth metal salts thereof, or mixtures of two or more thereof.

23. The method of claim 18, wherein the total amount of (ii) one or more hydroxylated (poly)carboxylic acids comprising from 2 to 8 carbon atoms, salts thereof, or mixtures thereof ranges from 0.5% to 10% by weight, relative to the total weight of the composition.

24. The method of claim 18, which the composition further comprises (iii) one or more non-ionic associative polymers, wherein the total amount of (iii) non-ionic associate polymer(s) ranges from 0.01% to 10% by weight, relative to the total weight of the composition.

25. The method of claim 18, wherein the composition further comprises (iv) one or more silicones, wherein the total amount of (iv) silicone(s) ranges from 0.3% to 5% by weight, relative to the total weight of the composition.

26. The method of claim 18, wherein the composition further comprises (v) one or more cationic surfactants, wherein the total amount of (v) cationic surfactant(s) ranges from 0.1% to 10% by weight, relative to the total weight of the composition.

27. The method of claim 18, wherein the composition further comprises (vi) one or more cationic polymers, wherein the total amount of (vi) cationic polymer(s) ranges from 0.01% to 10% by weight, relative to the total weight of the composition.

28. The method of claim 27, wherein the (vi) one or more cationic polymers is chosen from cationic celluloses, galactomannan gums, or mixtures thereof.

29. The method of claim 18, wherein the composition further comprises (vii) one or more non-ionic polysaccharides, derivatives thereof, or mixtures thereof, wherein the total amount of (vii) non-ionic polysaccharides, derivatives thereof, or mixtures thereof ranges from 0.01% to 10% by weight, relative to the total weight of the composition.

30. The method of claim 28, wherein the (vii) one or more non-ionic polysaccharides, derivatives thereof, or mixtures thereof is chosen from celluloses, galactomannans, non-ionic derivatives thereof, or mixtures of two or more thereof.

31. The method of claim 18, wherein the composition further comprises (viii) one or more non-ionic surfactants, wherein the total amount of (viii) non-ionic surfactant(s) ranges from 0.05% to 10% by weight, relative to the total weight of the composition.

32. The method of claim 18, wherein the composition further comprises (ix) one or more non-silicone fatty substance(s), wherein the total amount of (ix) non-silicone fatty substance(s) ranges from 0.1% to 20% by weight, relative to the total weight of the composition.

33. The method of claim 32, wherein the (ix) one or more non-silicone fatty substances is chosen from triglyceride oils of vegetable or synthetic origin, liquid esters of a fatty acid and/or of a fatty alcohol other than triglycerides, liquid C₆-C₁₈ hydrocarbons, solid fatty alcohols, solid esters of a fatty acid and/or of a fatty alcohol, or mixtures of two or more thereof.

34. The method of claim 18, wherein the composition further (x) comprises water, wherein the total amount of (x) water ranges from 50% to 95% by weight, relative to the total weight of the composition.

35. The method of claim 18, further comprising: (b) rinsing the hair with water or with a shampoo after an optional leave-on time.

36. The method of claim 18, wherein the method is a method for treating sensitized, weakened and/or damaged hair, or hair laden with calcium in an amount of more than 4000 ppm.

37. A method for limiting the calcium amount of the hair, comprising applying to the hair a composition comprising:
(i) one or more amino acid compounds of formula (I), salts thereof, or mixtures thereof: 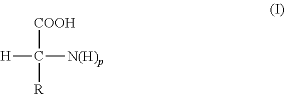
wherein p is an integer equal to 1 or 2,
wherein when p=1, R forms, with the nitrogen atom, a saturated heterocycle comprising from 5 to 8 ring members, substituted by one or more groups chosen from hydroxyl or (C₁-C₄)alkyl,
wherein when p=2, R represents a hydrogen atom or a saturated, linear or branched, (C₁-C₁₂)alkyl group, optionally interrupted by one or more heteroatoms or groups chosen from —S—, —NH— or —C(NH)—, and optionally substituted by one or more groups chosen from hydroxyl (—OH), amino (—NH₂), —SH, —COOH, —CONH₂ or —NH—C(NH)—NH₂; and
wherein the total amount of amino acid compounds of formula (I), salts thereof, or mixtures thereof is at least 0.5% by weight, relative to the total weight of the composition; and
(ii) one or more hydroxylated (poly)carboxylic acids comprising from 2 to 8 carbon atoms, salts thereof, or mixtures thereof, wherein the total amount of one or more hydroxylated (poly)carboxylic acids comprising from 2 to 8 carbon atoms, salts thereof, or mixtures thereof is at least 0.5% by weight, relative to the total weight of the composition.
